(19) United States
(12) Patent Application Publication
(10) Pub. No.: US 2012/0065153 A1
(43) Pub. Date: Mar. 15, 2012

(54) AZASUGAR DERIVATIVES, HEPARANASE INHIBITORS, METHOD FOR PREPARING SAME, COMPOSITIONS CONTAINING SAME, USE THEREOF

(75) Inventors: Maurice PETITOU, Paris (FR); Pierre Alexandre DRIGUEZ, Toulouse (FR)

(73) Assignee: SANOFI-AVENTIS, Paris (FR)

(21) Appl. No.: 13/227,922

(22) Filed: Sep. 8, 2011

Related U.S. Application Data
(63) Continuation of application No. 11/625,994, filed on Jan. 23, 2007.
Application No. 11/625,994 is a continuation of application No. PCT/FR2005/001851, filed on Jul. 20, 2005.

(30) Foreign Application Priority Data
Jul. 23, 2004 (FR) 0408160

(51) Int. Cl. A61K 31/706; A61P 35/00; A61P 35/04; A61P 35/02; A61P 29/00; A61P 27/02; A61P 1/00; A61P 1/04; A61P 9/00; A61P 13/12; A61P 7/00; A61P 9/10; C07H 17/02; A61P 37/00

(52) U.S. Cl. 514/27; 536/17.4

(57) ABSTRACT

The invention concerns heparanase inhibiting compounds of general formula (I) wherein R represents a hydrogen atom, an hydroxyl radical, a —OSO₃— radical, a —O—(C₁-C₅)alkyl radical or an —O— aralkyl radical; Z represents a COO⁻ radical or a hydroxyl radical; X represents —OH or a saccharide unit of formula A, Y represents H, C₁-C₅ alkyl or a saccharide unit of formula D; in free form or in the form of pharmaceutically acceptable salts formed with a base or an acid as well as in the form of solvates or hydrates. The derivatives of the invention are useful as medicines.

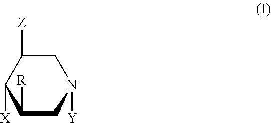

The present application is a continuation application of U.S. Ser. No. 11/625,994, filed Jan. 23, 2007, which is a continuation of PCT/FR2005/001851 filed Jul. 20, 2005, the entire contents of which are incorporated herein by reference.

The subject of the invention is azasugar derivatives, inhibitors of heparanases, their preparation, compositions containing them and their therapeutic application.

Heparanases are enzymes of the endoglucuronidase type which have as substrate polysaccharides of the heparin/heparan sulphate (HS) family. Type I and II heparanases are known (McKenzie et al., Biochem. Biophys. Res. Commun. (2000), Vol. 276, p. 1170-1177). They hydrolyse specifically β-1→4 type bonds between a saccharide unit of the D-glucuronic acid type and a saccharide unit of the D-glucosamine type and release HS fragments of about 10 to 20 saccharide units (Pikas, D. S. et al., J. Biol. Chem., (1998), Vol. 273, p. 18770-18777). Heparanases break down in the same manner the polysaccharide chains of heparan sulphate proteoglycans (HSPGs) (Vlokaysky and Friedmann, J. Clin Invest., (2001), Vol. 108, p. 341-347). HSPGs consist of a core protein to which linear HS chains are covalently attached (Kjellen et al., Annu. Rev. Biochem., (1991), Vol. 60, p. 443-475). HSPGs are ubiquitous macromolecules. Like HSs, HSPGs are present at the surface of numerous cell types and in the extracellular matrix (ECM) (Kjellen et al., (1991), ibid.; Bernfield et al., Annu Rev. Biochem., (1999), Vol. 68, p. 729-777; David et al., FASEB J., (1993), Vol. 7, p. 1023-1030; Lozzo et al., Annu. Rev. Biochem. (1998), Vol. 67, p. 609-652). The ECM, a major component of the connective tissues of vertebrates and invertebrates, occupies the extracellular environment. It envelops the organs and surrounds the endothelia, in particular the capillary endothelia (Wight et al., Arteriosclerosis, (1989), Vol. 9, p. 1-20), thus playing a role of maintenance and of barrier for protection of the organs and endothelia (McKenzie et al., Biochem. J.; 2003; Vol. 373, p. 423-435). The ECM is also a key modulator involved in various cellular mechanisms, in particular cell differentiation and repair (Folkman et al., Adv. Exp. Med. Biol., (1992), Vol. 313, p. 355-364).

Heparanase inhibiting compounds have been described in the prior art. For example, International Patent Application WO 02/0600374 describes benz-1,3-azole derivatives, International Patent Application WO 03/074516 relates to phthalimidecarboxylic acid and benzoxazole derivatives, International Patent Application WO 04/013132 describes furanthiazole derivatives or International Patent Application WO 04/046123 describes benzoxazole, benzothiazole and benzimidazole derivatives.

The synthesis of short-chain (2 units) azasugar derivatives in which the nitrogen atom replaces the oxygen atom at the 5-position has been described in Takahashi et al., Chem. Lett., (1994), Vol. 11, p. 2119; Takahashi et al., Tetrahedron, (2001), Vol. 57, p. 6915-6926). However, their activities in vivo have not been identified.

Azasugar derivatives with a single unit, of the following formula:

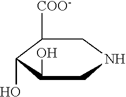

have already been described (U.S. Pat. No. 6,583,158; Ichikawa et al., J. Amer. Chem. Soc., (1998), Vol. 120, p. 3007).

A need still exists to find and to develop products having good activity in vitro and in vivo.

It has now been found, surprisingly, that synthetic azasugar derivatives exhibit good activity as heparanase inhibitors. The present invention therefore relates to novel azasugar derivatives which are heparanase inhibitors. These novel compounds exhibit good heparanase inhibiting activity.

The subject of the invention relates to compounds corresponding to the general formula (I):

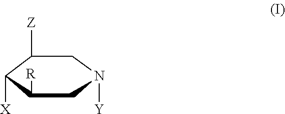

in which:
R represents a hydrogen atom, a hydroxyl radical, an —OSO₃⁻ radical, an —O—(C₁-C₅)alkyl radical or an O-aralkyl radical;
Z represents a COO⁻ radical or a hydroxyl radical;
X represents a hydroxyl radical or a saccharide unit of formula A:

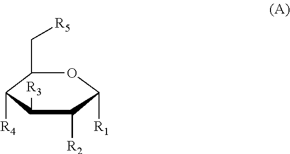

in which:
R₁ represents an oxygen atom, allowing A to bind to the azasugar unit or to another saccharide unit,
R₂ represents an —NH₂ radical, an —NHCO(C₁-C₅)-alkyl radical, an —NHCOaryl radical, an —NHSO₃⁻ radical, a hydroxyl radical, an —O—(C₁-C₅)alkyl radical, an —O-aralkyl radical or an —OSO₃⁻ radical,
R₃ represents a hydroxyl radical, an —OSO₃⁻ radical, an —O—(C₁-C₅)alkyl radical or an —O-aralkyl radical,
R₄ represents a hydroxyl radical, an —OSO₃⁻ radical, an —O—(C₁-C₅)alkyl radical, an O-aralkyl radical or a saccharide unit of formula B:

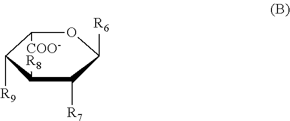

in which:
   R₆ represents an oxygen atom, allowing B to bind to another saccharide unit of formula A,
   R₇ and R₈ have the same definition as R₃ as defined above,
   R₉ represents a hydroxyl radical, an —OSO₃⁻ radical, an —O—(C₁-C₅)alkyl radical, an —O-aralkyl radical or a saccharide unit of formula A as defined above,
R₅ has the same definition as R₃ as defined above;
   Y represents a hydrogen atom, a (C₁-C₅)alkyl radical or a saccharide unit of formula D

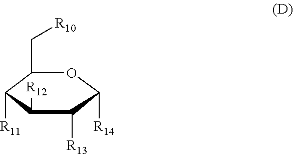

in which:
R₁₀, R₁₂ and R₁₃ have the same definitions as R₅, R₃ and R₂ respectively as defined above,
R₁₁ represents:
   a (C₁-C₃)alkylene radical allowing D to attach to the azasugar unit, or
   an oxygen atom allowing D to attach to another saccharide unit,
R₁₄ represents an —O—(C₁-C₅)alkyl radical or an —O-E radical in which E represents a radical of the following formula:

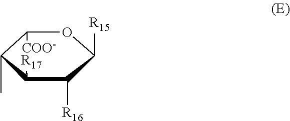

in which:
   R₁₅ represents an —O—(C₁-C₅)alkyl radical, an —O-aralkyl radical or a saccharide unit of formula D in which R₁₁ represents an oxygen atom,
   R₁₆ and R₁₇ have the same definition as R₃ as defined above, provided, however, that when X and R each represent a hydroxyl radical, Y does not represent a hydrogen atom, and it being understood that the number of saccharide units of which the compound of formula (I) is composed is between 1 and 10,
      in free form or in the form of salts formed with a pharmaceutically acceptable base or acid, and in the form of solvates or hydrates.

According to one of its preferred aspects, the invention relates to the compounds of general formula (I):

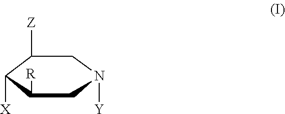

in which:
R represents a hydroxyl radical;
Z represents a COO⁻ radical or a hydroxyl radical;
X represents a hydroxyl radical or a saccharide unit of formula A:

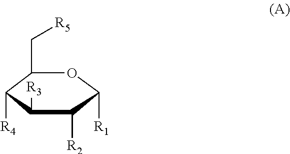

in which:
R₁ represents an oxygen atom,
R₂ represents an —NHCOCH₃ radical, an —NHSO₃⁻ radical, an —OSO₃⁻ radical,
R₃ represents a hydroxyl radical or an O—(C₁-C₅)alkyl radical,
R₄ represents a hydroxyl radical, an —O-aralkyl radical or a saccharide unit of formula B:

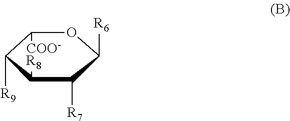

in which:
   R₆ represents an oxygen atom,
   R₇ represents an —OSO₃⁻ radical,
   R₈ represents a hydroxyl radical, an —O—(C₁-C₅)alkyl radical or an —O-aralkyl radical,
   R₉ represents an —OSO₃⁻ radical, an —O-aralkyl radical, an —O—(C₁-C₅)alkyl radical or a saccharide unit of formula A as defined above,
R₅ represents an —OSO₃⁻ radical;
   Y represents a hydrogen atom or a saccharide unit of formula D:

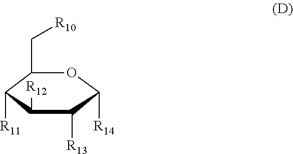

in which:
R₁₀ has the same definition as R₅ as defined above,
R₁₂ represents a hydroxyl radical or an —OSO₃⁻ radical,
R₁₃ represents an —NHSO₃⁻ radical,
R₁₁ represents a methylene radical linked to an azasugar unit or an oxygen atom linked to E,
R₁₄ an —OCH₃ radical or a radical of formula —O-E in which E represents a radical of formula:

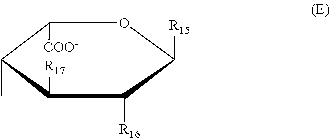

in which:
   R₁₅ represents a D unit in which R₁₁ represents an oxygen atom allowing E to be linked to D,
   R₁₆ represents an —OSO₃⁻ radical,
   R₁₇ represents a hydroxyl radical,
      it being understood that the number of saccharide units of which the compound of formula (I) is composed is between 2 and 10,
      in free form or in the form of salts with a pharmaceutically acceptable base or acid, and in the form of solvates or hydrates.

Particularly preferred compounds are compounds of formula I in which Y is a hydrogen atom.

The invention encompasses azasugar derivatives in their acid form or in the form of any one of their pharmaceutically acceptable salts. In the acid form, the —COO⁻ and —SO₃⁻ functional groups are in the —COOH and —SO₃H forms respectively.

According to one of its particularly preferred aspects, the present invention relates to the following compounds:
(2,4-di-O-sodium sulphonato-α-L-idopyranosyl-uronate of sodium)-(1-4)-(2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-3-carboxylate of sodium (3S, 4R, 5R)) (compound No. 20)
(2,4-di-O-sodium sulphonato-α-L-idopyranosyl-uronate of sodium)-(1-4)-(2-N-sodium sulphonato-2-deoxy-6-O-sodium sulphonato-α-D-gluco-pyranosyl)-(1-4)-(2-O-sodium sulphonato-α-L-idopyranosyluronate of sodium)-(1-4)-(2-N-sodium sulphonato-2-deoxy-6-O-sodium sulphonato-α-D-glucopyranosyl)-(1-4)-(5-(hydroxy)-4-oxypiperidine-3-carboxylate of sodium (3S, 4R, 5R)) (compound No. 27)
(3-O-methyl-2,4-di-O-sodium sulphonato-α-L-idopyranosyluronate of sodium)-(1-4)-(3-O-methyl-2,6-di-O-sodium sulphonato-α-D-gluco-pyranosyl)-(1-4)-(3-O-methyl-2-O-sodium sulphonato-α-L-idopyranosyluronate of sodium)-(1-4)-(3-O-methyl-2,6-di-O-sodium sulphonato-α-D-glucopyranosyl)-(1-4)-(5-(hydroxy)-4-oxypiperidine-3-carboxylate of sodium (3S, 4R, 5R)) (compound No. 47)
(2,4-di-O-sodium sulphonato-α-L-idopyranosyl-uronate of sodium)-(1-4)-(2,6)-di-O-sodium sulphonato-α-D-glucopyranosyl)-(1-4)-(2-O-sodium sulphonato-α-L-idopyranosyluronate of sodium)-(1-4)-(2,6-di-O-sodium sulphonato-α-D-gluco-pyranosyl)-(1-4)-(5-(hydroxy)-4-oxypiperidine-3-carboxylate of sodium (3S, 4R, 5R)) (compound No. 69)
(4-O-propyl-2-O-sodium sulphonato-α-L-idopyranosyluronate of sodium)-(1-4)-(2,6-di-O-sodium sulphonato-α-D-glucopyranosyl)-(1-4)-(2-O-sodium sulphonato-α-L-idopyranosyluronate of sodium)-(1-4)-(2,6-di-O-sodium sulphonato-α-D-glucopyranosyl)-(1-4)-(5-(hydroxy)-4-oxypiperidine-3-carboxylate of sodium (3S, 4R, 5R)) (compound No. 74)
(2,4-di-O-sodium sulphonato-α-L-idopyranosyluronate of sodium)-(1-4)-(2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1-4)-(3-(hydroxy)-5-hydroxymethyl-4-oxypiperidine (3R, 4R, 5R)) (compound No. 123)
(4-O-phenylpropyl-2-O-sodium sulphonato-α-L-idopyranosyluronate of sodium)-(1-4)-(2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-3-carboxylate of sodium (3S, 4R, 5R)) (compound No. 124).

In the context of the present invention:
a (C₁-C₅)alkyl radical represents a linear or branched saturated aliphatic radical which may contain from 1 to 5 carbon atoms. By way of examples, there may be mentioned methyl, ethyl, propyl, isopropyl, butyl, isobutyl and tert-butyl radicals and the like,
an —O-aralkyl radical represents an alkyl group as defined above but substituted with an aromatic radical which may itself carry substituents. By way of example, there may be mentioned p-methoxybenzyl, phenylmethyl, phenylethyl, phenylpropyl and the like,
a (C₁-C₃)alkylene radical represents a divalent alkyl group containing from 1 to 3 carbon atoms.

The expression pharmaceutically acceptable salt is understood to mean azasugar derivatives of the invention, an azasugar derivative in which one or more of the —COO⁻ or/and —SO₃⁻ functional groups are ionically linked to a pharmaceutically acceptable cation. The preferred salts according to the invention are those whose cation is chosen from alkali metal cations and still more preferably those whose cation is Na⁺ or K⁺.

In its principle, the method for preparing the compounds according to the invention uses mono-, di- or oligosaccharide parent synthons prepared as previously reported in the literature and chosen taking into account in particular the orthogonality of the protecting groups. Reference may be made in particular to EP 300099, EP 529715, EP 621282 and EP 649854 and to C. van Boeckel, M. Petitou, Angew. Chem. Int. Ed. Engl., (1993), Vol. 32, p. 1671-1690. These synthons are then coupled to each other so as to provide a completely protected equivalent of a compound according to the invention. This protected equivalent is then converted to a compound according to the invention using methods well known to the person skilled in the art.

As the compounds of the invention additionally contain an azasugar (or substituted piperidine) unit, their synthesis requires the preparation of a precursor of this unit carrying protecting groups compatible with subsequent couplings to mono-, di- or oligosaccharides. The azasugar precursors are prepared according to methods described in the literature. Reference may be made in particular to the book “Iminosugars as Glycosidase Inhibitors”, A E Stütz, Wiley-VCH, 1999.

When the synthons necessary for the assembly of the chain are available, couplings of these synthons with each other are carried out. In the coupling reactions mentioned, a “donor” synthon, activated on its anomeric carbon, reacts with an “acceptor” synthon, possessing at least one free hydroxyl.

The present invention relates to a method for the preparation of the compounds of formula (I),

characterized in that: in a first step, a completely protected equivalent of the desired compound (I) is prepared; in a second step, the negatively charged groups (carboxylates, sulphonates) and the free hydroxyls are introduced and/or unmasked.

The synthesis of the precursor is carried out according to reactions well known to persons skilled in the art using in particular the methods of saccharide synthesis (G. J. Boons, Tetrahedron, (1996), Vol. 52, p. 1095-1121; WO 98/03554 and WO 99/36443) according to which a glycosidic bond donor oligosaccharide is coupled with a glycosidic bond acceptor oligosaccharide to give another oligosaccharide whose size is equal to the sum of the sizes of the two reactive species. This sequence is repeated until the desired compound of formula (I) is obtained. The structure of the desired final compound determines the nature of the chemical entities used in the various steps of the synthesis, so as to control the stereochemistry and the regioselectivity, according to rules well known to persons skilled in the art.

The compounds according to the invention are obtained from their completely protected polysaccharide precursors using in general the following sequence of reactions:
release and substitution of the functional groups which have to be converted to N- and/or O-sulphonate groups;
release of the other functional groups and optional assembly of the fragments with each other, for example by a reductive amination reaction.
   The release of the carboxylic acid functional groups may be carried out at various stages of this method.

The compounds of the invention may naturally be prepared using various strategies known to persons skilled in the art for saccharide and organic synthesis.

The method described above is the preferred method of the invention. However, the compounds of formula (I) may be prepared by other methods well known in sugar chemistry which are described for example in “Monosaccharides, Their chemistry and their roles in natural products”, P. M. Collins and R. J. Ferrier, J. Wiley & Sons, 1995 and in G. J. Boons, Tetrahedron, (1996), Vol. 52, p. 1095-1121.

The protecting groups used in the method for preparing the compounds (I) are those commonly used in sugar chemistry, for example in Protective Groups in Organic Synthesis, T W Greene, P G M Wuts, John Wiley & Sons, New York, 1999.

The protecting groups are advantageously chosen for example from acetyl, halomethyl, benzoyl, levulinyl, benzyl, substituted benzyl, optionally substituted trityl, carbamate, tetrahydropyranyl, allyl, pentenyl, tert-butyldimethylsilyl (tBDMS) or trimethylsilylethyl groups.

The activating groups are those conventionally used in sugar chemistry according to for example G. J. Boons, Tetrahedron, (1996), Vol. 52, p. 1095-1121. These activating groups are chosen for example from imidates, thioglycosides, pentenyl-glycosides, xanthates, phosphites or halides.

The method described above makes it possible to obtain compounds of the invention in the form of salts. To obtain the corresponding acids, the compounds of the invention in the form of salts are brought into contact with a cation exchange resin in acid form.

The compounds of the invention in the form of acids may then be neutralized with a base in order to obtain the desired salt. For the preparation of the salts of the compounds of formula (I), it is possible to use any inorganic or organic base which gives pharmaceutically acceptable salts with the compounds of formula (I). Sodium, potassium, calcium or magnesium hydroxide is preferably used as base. The sodium and calcium salts of the compounds of formula (I) are the preferred salts.

The compounds according to the invention have been the subject of biochemical and pharmacological studies. The following nonlimiting tests illustrate the present invention.

The Following Terms are Defined:

PET: polyethylene terephthalate,
AM: acetoxymethyl,
DMEM: Dulbecco's modified Eagle's medium,
EDTA: ethylenediaminetetraacetic acid,
Tris: tris(hydroxymethyl)aminomethane,
AT: antithrombin III,
nkat: nanokatal=enzymatic unit of measurement (given by the manufacturer) representing the quantity of substrate catalysed per unit of time.

### 1. Evaluation of the Activity of the Heparanase Inhibitors in an Enzymatic System (Determination of the IC₅₀ Values of the Compounds According to the Invention)

The heparanase activity is demonstrated by its capacity to degrade fondaparinux. The concentration of fondaparinux is determined by means of its anti-factor Xa activity.

### A. Materials and Methods

The heparanase is produced by Sanofi-Synthelabo (Labège, France).

The reagents for assaying factor Xa are marketed by Chromogénix (Montpellier, France).

Increasing concentrations of a compound according to the invention, an inhibitor of heparanases (varying dilutions: from 1 nM to 10 μM) are mixed at a fixed heparanase concentration (for each batch, preliminary experiments make it possible to determine the enzymatic activity which is sufficient for the degradation of 0.45 μg/ml of added fondaparinux). After 5 minutes at 37° C., the mixture is exposed to fondaparinux and left for 1 hour at 37° C. The reaction is stopped by heating at 95° C. for 5 minutes. The residual fondaparinux concentration is finally measured by adding factor Xa and its specific chromogenic substrate (Ref. S2222).

The various mixtures are produced according to the following procedure:

a) Reaction Mixture

50 μl of sodium acetate buffer (0.2 M, pH 4.2) are mixed with 50 μl of fondaparinux (0.45 μg/ml) and 59 μl of a heparanase solution. The mixture is incubated for 1 hour at 37° C. and then for 5 minutes at 95° C. 100 μl of the reaction mixture are then mixed with 50 μl of 50 mM Tris buffer containing 175 mM NaCl, 75 mM EDTA, pH 14. The pH thus passes from 4.2 to 7.

The anti-factor Xa activity of fondaparinux is measured in the following manner:

b) Assay of the Anti-Factor Xa Activity of Fondaparinux

100 μl of the solution obtained in step a) are mixed with 100 μl of AT (0.5 μ/ml). The mixture is kept for 2 minutes at 37° C. and 100 μl of factor Xa (7 nkat/ml) are then added. The mixture is kept for 2 minutes at 37° C. and 100 μl of chromogenic substrate (Ref.: S2222) (1 mM) are then added. The mixture is kept for 2 minutes at 37° C. and 100 μl of acetic acid (50%) are then added.

The optical density is read at 405 nm.

A percentage inhibition is determined relative to control without inhibitor. A curve of the percentage inhibition makes it possible to calculate an IC₅₀.

### B. Results

The compounds according to the present invention have IC₅₀ values of between 10 nm and 10 μM.

For example, compound No. 27 has an IC₅₀ of 11±4 nM (mean±SD, done on two assays).

### 2. Effect of the Heparanase Inhibitors on the Invasion of HT1080 Tumour Cells

The effect of the compounds of formula (I), inhibitors of heparanases, was tested in vitro on the invasion of HT1080 tumour cells.

### A. Materials and Methods

a) Cell Culture:

The cells derived from human fibrosarcomas, HT1080 (ATCC CCL-121) are cultured in a DMEM medium (Ref.: GIBCO 11960-044) containing 5% Foetal Calf Serum, glutamine (2 mM) (Ref.: GIBCO 25030-024), sodium pyruvate (1 mM) (Ref.: GIBCO 11360-039) and nonessential amino acids (1×) (Ref.: GIBCO 11140-035), on collagen coated flasks (Becton Dickinson 75 cm²; Ref. 354523), to 50 to 80% confluence.

b) Cell Invasion Test

The measurements of invasion of the HT1080 cells are carried out on a Becton Dickinson falcon HTS Fluoroblock Multiwell Insert System kit in 24-well plates (Ref.: 351158). These measurement chambers give the cells the conditions which make it possible to evaluate their invasive properties in vitro.

The kit is composed of a plate combined with culture inserts containing a PET membrane pierced with 8 micron pores on which a uniform Matrigel Matrix (Becton Dickinson; Ref. 354230) layer is deposited.

Matrigel is a soluble basal membrane extracted from the EHS (Engelbreth-Holm-Swarm) tumour which, by virtue of its composition, forms, upon solidifying, a structure equivalent to a basal membrane.

The Matrigel layer blocks the pores of the membrane, thus blocking the migration of the noninvasive cells across the membrane. By contrast, the invasive (tumour or nontumour) cells will be capable of becoming detached and of invading the Matrigel layer before migrating across the membrane.

The quantification of the cell migration is carried out by labelling with calcein AM (Molecular Probes C-3100). The fluorescent signal emitted is measured with the aid of a Perkin Elmer Wallac VICTOR 3 reader and may be directly correlated with the number of cells which have invaded the Matrigel gel. By comparing them with controls made in the same experiment as the products studied (response in the presence of 0% and 5% foetal calf serum), it is possible to determine a percentage inhibition of cell invasion in the presence of the products.

### B. Results

A series of independent determinations (varying from 2 to 4) made it possible to show that at a concentration of between 1 nM and 10 μM, the compounds according to the invention inhibit cell invasion on average by a percentage of between 8 and 60%.

For example, a series of four independent determinations made it possible to show that 10 μM of compound No. 20 inhibit cell invasion on average by a percentage equal to 40.3±8.3% (mean±standard deviation).

Furthermore, compound No. 20 has a dose-dependent effect on cell invasion.

### Indeed:

at a concentration of 0.3 μM, compound No. 20 inhibits cell invasion by 26±3%,
at a concentration of 1 μM, compound No. 20 inhibits cell invasion by 53±11%.

These results demonstrate an increase in the inhibition of cell invasion as a function of the dose of compound No. 20.

The compounds of formula (I) according to the present invention therefore exhibit good affinity for heparanases and exhibit a heparanase inhibiting effect.

It has been demonstrated in animals and in humans that the increase in the secretion of heparanases and cancerous progression are correlated (Goldschmit et al., PNAS, (2002), Vol. 99 (15), p. 10031-10036). For example, a high heparanase level has been detected in the serum of animals having metastatic tumours (Vlodaysky et al., Isr. J. Med. Sci. (1988), Vol. 24 (9-10), p. 464-470) or in the urine of patients suffering from cancer who have developed numerous metastases (Vlodaysky et al., Curr. Biol., (1997), Vol. 7 (1), p. 43-51). Tumour biopsies have shown the same correlation (Vlodaysky et al., Isr. J. Med. Sci., (1988), Vol. 24 (9-10), p. 464-470). A correlation therefore exists between the increase in secretion of heparanases and the metastatic potential of tumour cells (Vlodaysky et al., Invasion Metastasis, (1994), Vol. 14, p. 290-302; Nature Medicine, (1999), Vol. 5, p. 793-802).

Heparanases, which are secreted by tumour cells, degrade the HSPGs and HS, which are major components of the ECM. The ECM thus perforated allows the tumour and metastatic cells to circulate and also allows the invasion of newly-formed blood vessels (angiogenesis) (Suzanne A. Eccles, Nat. Med., (1999), Vol. 5 (7), p. 793-809). Angiogenesis is a process for generating new capillary vessels from preexisting vessels or by mobilization and differentiation of bone marrow cells. Thus, both an uncontrolled proliferation of the endothelial cells and a mobilization of angioblasts from the bone marrow are observed in the new vascularization processes of tumours.

Thus, heparanases represent relevant targets for therapies aimed at inhibiting the processes of invasion of cancer cells and of metastasization, on the one hand, and of angiogenesis, on the other. The expression cancer (or carcinoma) is understood to mean any malignant cell growth of the epithelium, present in the skin but also and especially in the wall of the organs and the appearance of metastatic tumour cells such as melanomas, mesothelioma, lymphoma, leukaemia, fibrosarcoma, rhabdomyosarcoma, mastocytoma, but also carcinomas affecting a tissue such as the colon, the rectum, the prostate, the lungs, the breasts, the pancreas, the intestine, the kidneys, the ovaries, the uterus, the cervix, the bladder, the liver and the stomach. The carcinomas infiltrate the adjacent tissues and spread (metastasize) to other distant organs, for example the liver, the lungs, the brain or the bones. A compound possessing a heparanase inhibiting activity, such as the compounds of the invention, may therefore be useful for the treatment of such cancers (Fang J et al., Proc. Natl. Acad. Sci. USA, (2000), Vol. 97 (8), p. 3884-3889; Kondraganti et al., Cancer Res., (2000), Vol. 60 (24), p. 6851-6855), and in particular colorectal, prostate, lung, breast, pancreatic, kidney, bladder and ovarian cancer.

The p75 receptor, a receptor for molecules of the neurotrophin (NT) family, has been identified as being a representative marker of the phenomenon of metastasization in the brain. It has furthermore been demonstrated that the secretion of NT involves an increase in the secretion of heparanases (Marchetti D. et al. J. Cell. Biochem., (2004), Vol. 91 (1), p. 206-215). Thus, a compound possessing a heparanase inhibiting activity, such as the compounds of the invention, may therefore be useful for reducing metastasization in the central nervous system by inhibiting the action of the activation of the p75 receptor (Marchetti D. et al., Pathol. Oncol. Res., (2003) Vol. 9 (3), p. 147-158 and Epub, (2003), Review; Walch E T. et al., Int. J. Cancer, (1999), Vol. 82 (1), p. 112-120; Menter D G. et al., Invasion Metastasis, (1994-95), Vol. 14 (1-6), p. 372-384; Marchetti D. et al., Int. J. Cancer, (1993), Vol. 55 (4), p. 692-699).

The activity of heparanases on the HSPGs of the ECM also appears to be correlated with the onset of inflammatory and autoimmune reactions (Vlodaysky et al., Invasion metastasis, (1994), Vol. 4, p. 290-302; Goldschmit et al., Med. Sci. (2002), Vol. 99 (15), p. 10031-10036). The interaction of the platelets, the granulocytes, the B and T lymphocytes, the macrophages and the mastocytes with the ECM is associated with the degradation of the HSs by the heparanases (Vlodaysky et al., Invasion Metastasis, (1992), Vol. 12, p. 112-127). Thus, a compound such as the compounds of the invention, possessing a heparanase inhibiting activity, may therefore be useful for the treatment of inflammatory diseases, in particular chronic inflammatory diseases such as rheumatoid arthritis or IBD (Inflammatory Bowel Disease), comprising two forms of chronic inflammatory bowel diseases: UC (ulcerative colitis) and Crohn's disease (CD) or autoimmune diseases.

Other studies suggest that heparanases could play a role in the treatment of cardiovascular diseases (Journal of Pharmacological Sciences, (2004), 94, No. Supplement 1, pp. 160P, print.; and Meeting Info.: 77th Annual Meeting of the Japanese Pharmacological Society, Osaka, Japan. Mar. 8-10, 2004. Japanese Pharmacological Society; and Miller, Heather Ann, Diss. Abstr. Int., (1984), B 2003, 64(5); Demir et al., Clin. Appl. Thromb. Hemost., (2001), Vol. 7 (2), p. 131-140), such as post-angioplasty restenosis, diseases linked to the vascular complications of diabetes such as diabetic retinopathies, atherosclerosis (Atherosclerosis, (1999), Vol. 145, p. 97-106; J. Clin. Invest., (1997), Vol. 100, p. 867-874) or thromboembolic diseases and arterial thromboses. Thus, a heparanase inhibiting compound according to the invention may represent a therapy of choice in these pathologies.

Moreover, heparanases are known to be involved in certain cases of renal insufficiency where the renal filtration and reabsorption functions may be impaired (FASEB J, (2004), Vol. 18, p. 252-263). Thus, a heparanase inhibiting compound according to the invention may represent a therapy of choice in such pathologies.

The HSPGs of the ECM also appear to play a role as major regulators of cell growth and activation via the modulation of growth factors, in particular of FGFs (Fibroblast Growth Factors). For example, the activity of heparanases involves the release of growth factors such as the FGFs, which stimulate in particular angiogenesis, and promotes tumour progression (Bashkin et al., Biochemistry, (1989), Vol. 28, p. 1737-1743). Thus, heparanases represent relevant targets for the treatment of diseases in which the FGFs are involved.

In general, the FGFs are involved to a great extent via autocrine, paracrine or juxtacrine secretions in the phenomena of deregulation of the stimulation of the growth of cancer cells. Furthermore, the FGFs initiate tumour angiogenesis which plays a predominant role both on tumour growth and alto on the phenomena of metastasization.

Angiogenesis is a process for generating new capillary vessels from preexisting vessels or by mobilization and differentiation of bone marrow cells. Thus, both uncontrolled proliferation of the endothelial cells and mobilization of angioblasts from the bone marrow are observed in the processes of neovascularization of tumours. It has been shown in vitro and in vivo that several growth factors stimulate endothelial proliferation, and in particular FGF-1 or a-FGF and FGF-2 or b-FGF.

a-FGF and b-FGF play for example an important role in the growth and the maintenance of prostate cells (Doll J A, et al., Prostate, (2001), Vol. 305, p. 49-293.

Several research studies show the presence of a-FGF and b-FGF and of their receptors (FGFRs) both in human breast tumour lines (in particular MCF7) and in tumour biopsies.

Glioma cells produce a-FGF and b-FGF in vitro and in vivo and possess various FGF receptors at their surface.

More recently, the potential role of proangiogenic agents, and in particular of FGFs, in leukaemias and lymphomas has been documented (Thomas D A et al., Acta Haematol, (2001), Vol. 207, p. 106-190).

A correlation exists between the process of bone marrow angiogenesis and extramedullar diseases in CMLs (chronic myelomonocytic leukaemia).

The proliferation and migration of vascular smooth muscle cells contribute to the intimal hypertrophy of the arteries and thus plays a predominant role in atherosclerosis and in restenosis following angioplasty and endarterectomy. Studies in vivo show a local production of a-FGF and b-FGF after lesion of the carotid by balloon injury.

Vascular disorders due to diabetes are characterized by an impairment of the vascular reactivity and of the blood flow, a hyperpermeability, an increased proliferative response and an increase in matrix protein deposits. More precisely, a-FGF and b-FGF are present in the preretinal membranes of patients with diabetic retinopathies, in the membranes of the underlying capillaries and in the vitreous humour of patients suffering from proliferative retinopathies.

Rheumatoid arthritis (RA) is a chronic disease with unknown etiology. While it affects numerous organs, the most severe form of RA is a progressive synovial inflammation of the joints resulting in destruction. Angiogenesis appears to greatly affect the progression of this pathology. Thus, a-FGF and b-FGF have been detected in the synovial tissue and in the joint fluid of patients suffering from RA, indicating that this growth factor is involved in the initiation and/or the progression of this pathology. In AIA models (adjuvant-induced model of arthritis) in rats, it has been shown that the overexpression of b-FGF increases the severity of the disease whereas an anti b-FGF neutralizing antibody blocks the progression of RA (Yamashita et al., J. Immunol., (2002), Vol. 57, pl 168-450; Manabe et al., Rheumatol, (1999), Vol. 20, p. 38-714).

Angiogenesis and inflammation are also major phenomena which occur in the processes involved in osteoarthritis leading to destruction of the joint accompanied by pain. Angiogenesis may also play a role in chondrocytic hypertrophy and ossification, thus contributing to the modifications of the joint (Bonnet C S et al, Rheumatology. (1) 7-16 2005).

IBDs (inflammatory bowel diseases) comprise two forms of chronic inflammatory bowel diseases: UC (ulcerative colitis) and Crohn's disease (CD). IBDs are characterized by an immune dysfunction resulting in an inappropriate production of inflammatory cytokines inducing the establishment of a local microvascular system. The consequence of this angiogenesis of inflammatory origin is an intestinal ischaemia induced by vasoconstriction. High circulating and local b-FGF levels have been measured in patients suffering from these pathologies (Kanazawa et al., American Journal of Gastroenterology, (2001), Vol. 28, p. 96-822; Thorn et al., Scandinavian Journal of Gastroenterology, (2000), Vol. 12, p. 35-408).

A compound possessing a heparanase inhibiting activity, such as the compounds of the invention, may therefore be useful for the treatment of diseases linked to an up regulation of the FGFs and/or of their receptors.

By virtue of their low toxicity and their pharmacological and biological properties, the compounds of the present invention find application in the treatment of any carcinoma having a high degree of vascularization (lung, breast, prostate, oesophagus) or inducing metastases (colon, stomach, melanoma) or sensitive to a-FGF or to b-FGF in an autocrine manner or finally in pathologies of the lymphoma and leukaemia type. These compounds represent a therapy of choice either alone or in combination with a suitable chemotherapy or radiotherapy or in combination with a treatment with antiangiogenic agents. The compounds according to the invention also find application in the treatment of cardiovascular diseases such as atherosclerosis, post-angioplasty restenosis, in the treatment of diseases linked to the complications which appear following the fitting of endovascular prostheses and/or aortocoronary bypass surgery or vascular complications of diabetes such as diabetic retinopathies. The compounds of the invention also find application in the treatment of chronic inflammatory diseases such as rheumatoid arthritis or IBDs.

The products according to the invention also find application in the treatment of macular degeneration. A major characteristic of the loss of vision in adults is the neovascularization and the subsequent haemorrhages which cause considerable functional disorders in the eye and which result in early blindness. Recently, the study of the mechanisms involved in the phenomena of ocular neovascularization has made it possible to demonstrate the involvement of the proangiogenic factor in these pathologies.

The compounds of the invention may also be used in combination with one or more anticancer treatments, such as surgical treatments, radiotherapy or in combination with compounds which block angiogenesis. For example, the compounds of the invention may be used alone or in combination with another active ingredient such as cisplatine, cyclophosphamide, methotrexate, 5-fluorouracil, paclitaxel, docetaxel, vincristine, vinblastine, vinorelbine, doxorubicin, tamoxifen, toremifene, megestrol acetate, anastrozole, goserelin, capecitabine and raloxifene or molecules having an antiangiogenic activity, for the treatment of cancer.

According to another of its aspects, the subject of the present invention is therefore a pharmaceutical composition containing, as active ingredient, a compound of formula (I) in free form or in the form of salts formed with a pharmaceutically acceptable base or acid, according to the invention, optionally in combination with one or more inert and appropriate excipients.

The said excipients are chosen according to the desired pharmaceutical dosage form and mode of administration: oral, sublingual, subcutaneous, intramuscular, intravenous, transdermal, transmucosal, local or rectal.

In each dosage unit, the active ingredient is present in quantities appropriate for the daily doses envisaged in order to obtain the desired prophylactic or therapeutic effect. Each dosage unit may contain from 0.1 to 100 mg of active ingredient, preferably 0.5 to 50 mg.

The pharmaceutical compositions of the invention may be intended for oral, sublingual, subcutaneous, intramuscular, intravenous, intratracheal, topical, intranasal, transdermal, rectal, intraocular and vaginal administration. The unit forms for administration may be for example tablets, gelatin capsules, granules, powders, oral or injectable solution or suspensions, patches, injector pens and suppositories. For local administration, ointments, creams, lotions, eyedrops, gels, sprays and oil may be envisaged.

The said unit forms contain doses which allow a daily administration of 1 to 100 mg of active ingredient per kg of body weight, according to the galenic form used.

To prepare tablets, a pharmaceutical vehicle which may be composed of diluents, such as, for example, lactose, microcrystalline cellulose and starch, and formulation adjuvants, such as binders (polyvinylpyrrolidone, hydroxypropylmethylcellulose, and the like), glidants such as silica, and lubricants such as magnesium stearate, stearic acid, glyceryl tribehenate and sodium stearylfumarate, is added to the active ingredient, micronized or not. Wetting agents or surfactants such as sodium lauryl sulphate may also be added.

The production techniques may be direct compression, dry granulation, wet granulation or hot-melt.

The tablets may be uncoated, coated for example with sucrose, or coated with various polymers or other appropriate materials. They may be designed to allow rapid, delayed or prolonged release of the active ingredient by virtue of the polymer matrices or the specific polymers used in the coating.

To prepare gelatin capsules, the active ingredient is mixed with dry pharmaceutical vehicles (simple mixture, dry or wet granulation, or hot-melt), liquid pharmaceutical vehicles or semi-solid pharmaceutical vehicles.

The gelatin capsules may be hard or soft, film-coated or not, so as to have a rapid, prolonged or delayed activity (for example for an enteric form).

A composition in syrup or elixir form or for administration in the form of droplets may contain the active ingredient together with a sweetener, preferably a calorie-free sweetener, methylparaben or propylparaben as antiseptic, a saliva modifier and a colouring.

The water-dispersible powders and granules may contain the active ingredient in the form of a mixture with dispersing agents or wetting agents, or dispersing agents such as polyvinylpyrrolidone, and with sweeteners and flavour corrigents.

For rectal administration, suppositories prepared with binders which melt at the rectal temperature, for example cocoa butter or polyethylene glycols, are used.

For parenteral administration, aqueous suspensions, isotonic salt solutions or injectable sterile solutions containing pharmacologically compatible dispersing agents and/or wetting agents, for example propylene glycol or butylene glycol, are used.

The active ingredient may also be formulated in the form of microcapsules, optionally with one or more carriers or additives, or with a polymer matrix or with a cyclodextrin (patches, prolonged-release forms).

The compositions for local administration according to the invention comprise a medium compatible with the skin. They may be provided in particular in the form of aqueous, alcoholic or aqueous-alcoholic solutions, gels, water-in-oil or oil-in-water emulsions having the appearance of a cream or a gel, microemulsions, aerosols, or even in the form of vesicular dispersions containing ionic and/or nonionic lipids. These galenic forms are prepared according to the customary methods of the fields considered.

The active ingredient may also be formulated in the form of microcapsules, optionally with one or more carriers or additives, or with a polymer matrix or with a cyclodextrin (patches, prolonged-release forms).

The local compositions according to the invention comprise a medium compatible with the skin. They may be provided in particular in the form of aqueous, alcoholic or aqueous-alcoholic solutions, gels, water-in-oil or oil-in-water emulsions having the appearance of a cream or of a gel, microemulsions, aerosols, or in the form of vesicular dispersions containing ionic and/or nonionic lipids. These galenic forms are prepared according to the customary methods of the fields considered.

The examples which follow, given without limitation, illustrate the preparation of compounds according to the present invention.

Abbreviations used in the text which follows:

Ac=acetyl
All=allyl
Bn=benzyl
Me=methyl
Ph=phenyl
PMB=(4-methoxy)benzyl
PMBBr=(3-bromo-4-methoxy)benzyl
Z=benzyloxycarbonyl
TLC=thin-layer chromatography

The examples which follow illustrate the preparation of compounds according to the invention, without limiting it. Before embarking on the preparation of these various examples, there is described below the preparation of compounds (PREPARATION) useful for the production of compounds of the invention or for other preparations.

For the preparations and examples which follow, the following experimental methods are used:

### Method 1

Oxidation of the Primary Alcohols to an Acid, and then Conversion to a Benzyl Ester

TEMPO® (0.02 molar equivalent) and a saturated aqueous sodium hydrogen carbonate solution (4 l/mol) are added to a solution of compound to be oxidized (1 molar equivalent) in tetrahydrofuran (THF) (3.5 l/mol). After cooling to 0° C., Bromodan (2 molar equivalents) is added dropwise over 20 min. After 3 h of magnetic stirring, the reaction mixture is concentrated and the residue is dried by repeated evaporation of dimethylformamide (DMF) (4.95 l/mol). The crude compound thus obtained is used as it is in the next step.

A solution of the preceding compound in dimethylformamide (13.1 l/mol) is treated at room temperature (1-15 h) with benzyl bromide (10 molar equivalents), and potassium hydrogen carbonate (5 molar equivalents). The reaction mixture is concentrated and then the residue is dissolved in ethyl acetate (35 l/mol), washed with water, dried (sodium sulphate) and concentrated. Column chromatography gives the expected benzyl ester.

### Method 2

Coupling to Imidates Catalysed by tert-butyldimethyl-silyl triflate

A solution of tert-butyldimethylsilyl triflate in dichloromethane (0.1M, 0.15 mol per mole of imidate.) is added, under argon, at −20° C., to a solution of the imidate and of the glycosyl acceptor in a dichloromethane/diethyl ether mixture (1:2, 22.5-45 l/mol) in the presence of 4 Å molecular sieves. After 10-45 minutes (TLC), solid sodium hydrogen carbonate is added. The solution is filtered, washed with water, dried and evaporated to dryness.

### Method 3

### Method for Saponification of the Esters

Hydrogen peroxide (H₂O₂) at 30% (7.16 l/mol ester) and an aqueous 0.7N lithium hydroxide solution (2.3 mol per mole of ester) are successively added, at −5° C., to a solution of compound to be saponified in tetrahydrofuran (1601/mol). After stirring for 1 h at −5° C., the reaction medium is placed for 4 h at 0° C. and then stirred at room temperature until the esters have been consumed. The crude reaction product is then optionally purified on an LH-20 column.

### Method 4

### Sulphonation

Triethylamine/sulphur trioxide complex (5 mol per hydroxyl functional group) is added to a solution in dimethylformamide (90 l/mol) of the compound to be sulphated. After 12 to 22 hours at 55° C., methanol or an aqueous sodium hydrogen carbonate solution is added at 0° C., and after stirring for 0.5-24 h at room temperature, the reaction medium is purified with the aid of an LH-20 column, or of two Sephadex® G-25 columns (eluted successively with a 0.2M aqueous sodium chloride solution, and then with water). The fractions containing the product are then concentrated under a high vacuum to give the desired product.

### Method 5

Hydrogenolysis of the Benzyl Ethers and/or of the Benzyl Esters

A solution of the compound in glacial acetic acid/water/tert-butanol mixture is kept stirring for 6-16 h (TLC) under a hydrogen atmosphere (3-15 bar) in the presence of 5 or 10% palladium on carbon (equivalent to 0.7-3 times the mass of the compound). After filtration, the solution is deposited at the top of a Sephadex® G-25 column, eluted with 0.2M sodium chloride. The fractions containing the product are concentrated and desalted using the same column eluted with water. The final compound is obtained after freeze-drying.

Preparations useful for producing the compounds according to the invention are described below.

### Preparation 1

### Synthesis of (3S,4R,5R)-dibenzyl 5-(benzyloxy)-4-hydroxypiperidine-1,3-dicarboxylate (No. 6)

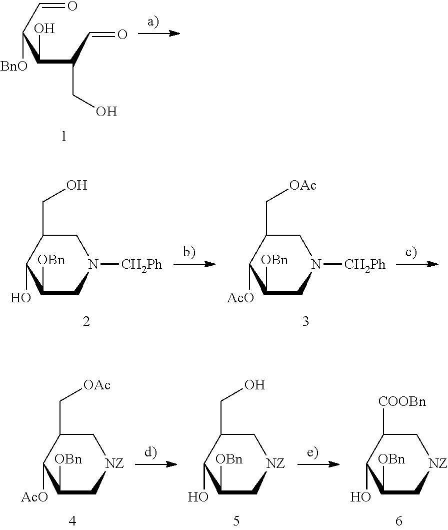

### Step 1.a: Preparation of (3R,4R,5R)-1-benzyl-3-(benzyloxy)-5-(hydroxymethyl)piperidin-4-ol (No. 2)

The synthesis of compound 1 is described in T. M. Jespersen and M. Bois, Tetrahedron (1994) 50 (47), 13449-13460 and in U.S. Pat. No. 5,844,102. The synthesis of compound 2 is described in Patent WO 98/50359.

To a solution of compound 1 (10.8 g, 42.8 mmol) in methanol (590 ml) are successively added sodium cyanoborohydride (5.38 g, 2 molar equivalents), followed by acetic acid (7.4 ml, 3 molar equivalents) at −10° C. and a benzylamine solution (5.1 ml, 1.1 equivalent) in methanol (100 ml). After returning to room temperature, the reaction mixture is heated at 50° C. for 2 h. After returning to room temperature, a 2% sodium hydrogen carbonate solution (85 ml) is added. The methanol is concentrated under vacuum and then the residue is diluted with dichloromethane and the organic phase is washed with water and then with an aqueous sodium chloride solution, dried (Na₂SO₄) and then concentrated under vacuum. The residue is used directly in the next step without purification.

### Step 1.b: Preparation of (3R,4R,5R)-methyl [4-(acetyl-oxy)-1-benzyl-5-(benzyloxy)piperidin-3-yl]acetate (No. 3)

Triethylamine (13.5 ml, 2.25 molar equivalents), 4-(dimethylamino)pyridine (DMAP) (7.84 g, 1.5 equivalents and acetic anhydride (61 ml, 15 molar equivalents) are successively added to a solution of the crude compound 2 (10.8 g) obtained in step 1.a in dichloromethane (345 ml). The temperature is kept at 0° C. for 10 min and then the reaction medium is placed at room temperature for 16 h. The reaction mixture is then concentrated under vacuum and the residue purified on silica gel to give compound 3 (7.65 g, 43%, 2 steps).

¹H NMR (CDCl₃) δ 7.36-7.18 (m, 10H, Ar), 4.60-4.42 (dd, 2H, OCH₂Ph), 2.02, 1.99 (2s, 6H, 2CH₃CO).

### Step 1.c: Preparation of (3R,4R,5R)-benzyl 4-(acetyl-oxy)-3-acetyloxymethyl)-5-(benzyloxy)piperidine-1-carboxylate (No. 4)

Benzyloxycarbonyl chloride (2.4 ml, 3 molar 25′ equivalents) is added under argon, at −10° C., to a solution of compound 3 (2.31 g, 5.6 mmol) obtained in step 1.b in tetrahydrofuran (28 ml), and then the reaction medium is left stirring at room temperature for 18 h. The reaction mixture is then concentrated under vacuum and the residue is purified on silica gel (1:9 diethyl ether-diisopropyl ether) to give compound 4 (2.14 g, 84%).

Mass spectrum (ESI) m/z 478.3 [(M+Na)⁺].

### Step 1.d: Preparation of (3R,4R,5R)-benzyl 3-(benzyl-oxy)-4-hydroxy-5-(hydroxymethyl)piperidine-1-carboxylate (No. 5)

A solution of 0.84M lithium hydroxide monohydrate (25 ml, 5 molar equivalents) is added, at 0° C., to a solution of compound 4 (1.9 g, 4.2 mmol) obtained in step 1.c, in dioxane (25 ml). The reaction medium is kept at 0° C. for 5 minutes and is then placed at room temperature for 30 min. After neutralizing with hydrochloric acid (HCl:3 N), the reaction medium is diluted in dichloromethane, washed with water, dried (Na₂SO₄), filtered and concentrated. The residue is purified on silica gel (3:1 ethyl acetate-cyclohexane) to give compound 5 (1.59 g, 90%)

Mass spectrum (ESI) m/z 394.4 [(M+Na)⁺].

### Step 1.e: Preparation of (3S,4R,5R)-dibenzyl 5-(benzyloxy)-4-hydroxypiperidine-1,3-dicarboxylate (No. 6)

Compound 5 (3.18 g, 8.6 mmol) obtained in step 1.d is treated according to METHOD 1 to give compound 6 (2.92 g, 71%).

Mass spectrum (ESI) m/z 476.5 [(M+Na)⁺].

### Preparation 2

### Synthesis of (benzyl 2,4-di-O-acetyl-3-O-benzyl-α-L-idopyranosyluronate)-(1-4)-(6-O-acetyl-2-azido-3-O-benzyl-2-deoxy-α,β-D-glucopyranose trichloro-acetimidate (No. 11)

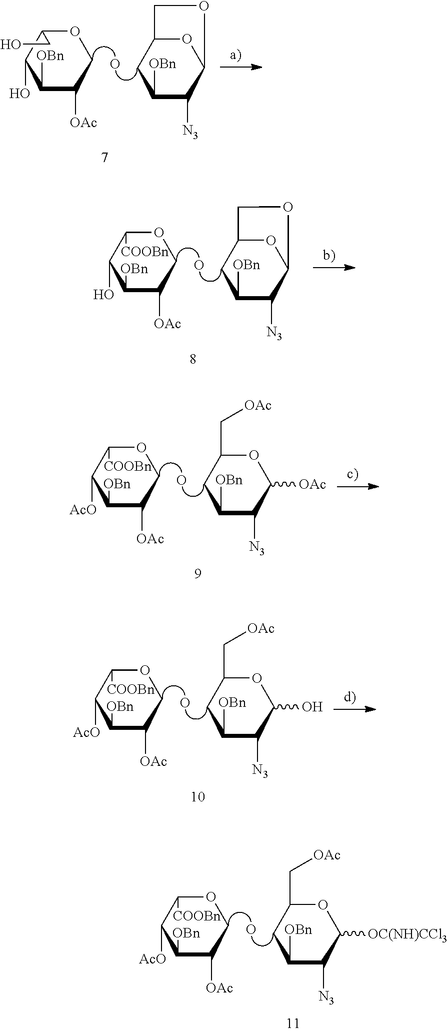

### Step 2.a: Preparation of (benzyl 2-O-acetyl-3-O-benzyl-α-L-idopyranosyluronate)-(1-4)-(1,6-anhydro-2-azido-3-O-benzyl-2-deoxy-β-D-glucopyranose) (No. 8)

Compound 7 in 2′-O-acetylated form (18.0 g, 31.48 mmol), prepared in the same manner as the 2′-O-benzoylated compound described in Y. Ichikawa et al., Tetrahedron Lett. (1986) 27 (5) 611-614, is treated according to METHOD 1 to give, after purification on silica gel (3:7 ethyl acetate-cyclohexane), compound 8 (16.4 g, 77%).

Mass spectrum (ESI) m/z 698.3 [(M+Na)⁺]

### Step 2.b: Preparation of (benzyl 2,4-di-O-acetyl-3-O-benzyl-α-L-idopyranosyluronate)-(1-4)-(1,6-di-O-acetyl-2-azido-3-O-benzyl-2-deoxy-α,β-D-glucopyranose) (No. 9)

Trifluoroacetic acid (TFA) (4.7 ml, 11 molar equivalents) is added, at 0° C., to a solution of compound 8 (3.74 g, 5.54 mmol) obtained in step 2.a, in acetic anhydride (52 ml, 100 molar equivalents). After returning to room temperature, the reaction mixture is stirred for 16 h and is then concentrated, coevaporated with toluene and purified on silica gel (4:1 toluene-ethyl acetate) to give compound 9 (4.33 g, 95%).

Mass spectrum (ESI) m/z 842.2 [(M+Na)⁺].

### Step 2.c: Preparation of (benzyl 2,4-di-O-acetyl-3-O-benzyl-α-L-idopyranosyluronate)-(1-4)-(6-O-acetyl-2-azido-3-O-benzyl-2-deoxy-α,β-D-glucopyranose) (No. 10)

Benzylamine (BnNH₂) (22 ml, 38 molar equivalents) is added, at 0° C., to a solution of compound 9 (4.3 g, 5.24 mmol) obtained in step 2.b, in diethyl ether (210 ml). After stirring for 4.5 h at room temperature, the medium is acidified with 1N HCl and is then extracted with ethyl acetate, dried (Na₂SO₄), concentrated and purified on silica gel (35:65 ethyl acetate-cyclohexane) to give 10 (3.4 g, 83%). Mass spectrum (ESI) m/z 800.2 [(M+Na)⁺].

### Step 2.d: Preparation of (benzyl 2,4-di-O-acetyl-3-O-benzyl-α-L-idopyranosyluronate)-(1-4)-(6-O-acetyl-2-azido-3-O-benzyl-2-deoxy-α,β-D-glucopyranose trichloroacetimidate) (No. 11)

Caesium carbonate (Cs₂CO₃) (2.26 g, 1.6 molar equivalents) and then trichloroacetonitrile (CCl₃CN) (1.74 ml, 5.0 molar equivalents) are added, under argon, to a solution of compound 10 (3.38 g, 4.35 mmol) obtained in step 2.c, in dichloromethane (82 ml). After stirring for 1.5 h, the reaction mixture is filtered and then concentrated. The residue is purified on silica gel (3:7 ethyl acetate-cyclohexane) to give 11 (2.96 g, 74%).

¹H NMR (CDCl₃) δ 6.43 (d, H−1α Glc^{I}), 5.64 (d, H−1β Glc^{I}), 5.17 (d, IdoUA^{II}).

### Preparation 3

### Synthesis of (3-O-benzyl-2,4-di-O-sodium sulphonato-α-L-idopyranosyluronate of sodium)-(1-4)-(2-acetamido-3-O-benzyl-2-deoxy-6-O-sodium sulphonato-α-D-glucopyranosyl)-(1-4)-(3-O-benzyl-2-O-sodium sulphonato-α-L-idopyranosyluronate of sodium)-(1-4)-(2-acetamido-3-O-benzyl-2-deoxy-6-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 19)

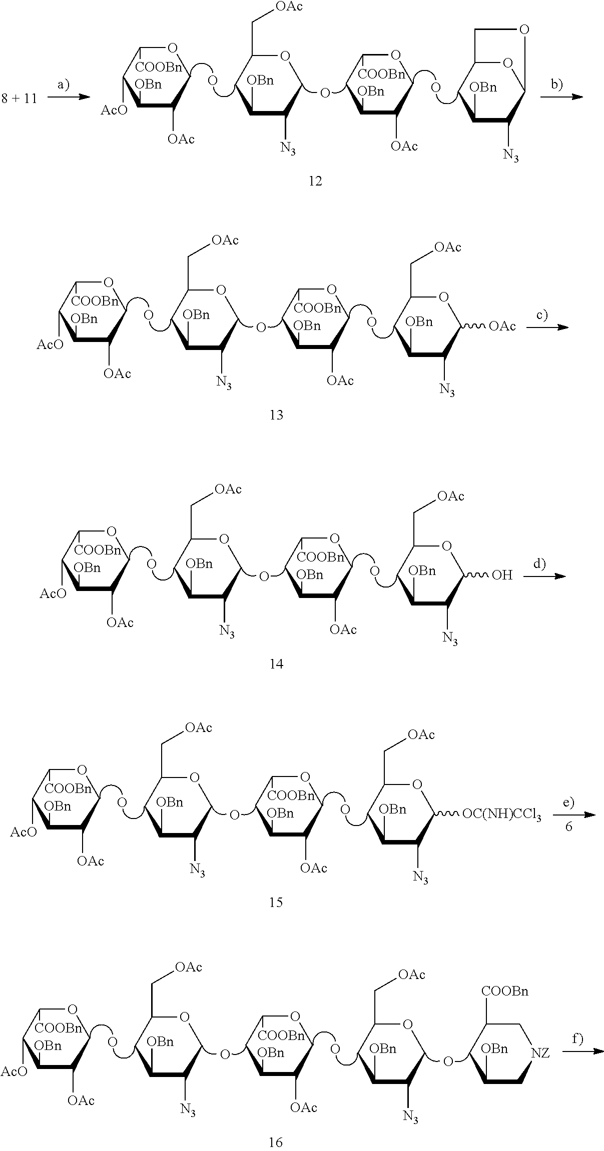

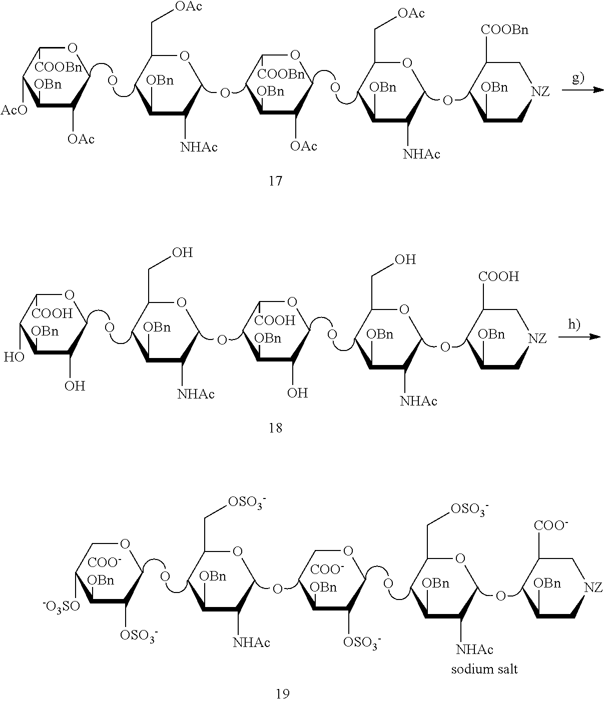

### Step 3.a: Preparation of (benzyl 2,4-di-O-acetyl-3-O-benzyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(1,6-anhydro-2-azido-3-O-benzyl-2-deoxy-β-D-glucopyranose) (No. 12)

Compound 11 (455 mg, 0.50 mmol) obtained in step 2.d and compound 8 (675 mg, 1 mmol) obtained in step 2.a are treated according to method 2 to give, after purification, compound 12 (385 mg, 54%). Mass spectrum (ESI) m/z 1457.6 [(M+Na)].

### Step 3.b: Preparation of (benzyl 2,4-di-O-acetyl-3-O-benzyl-α-L-idopyranosyluronate)-(1-4)-(6-O-acetyl-2-azido-3-O-benzyl-2-deoxy-α-D-glucopyranosyluronate-(1-4)-(benzyl 2-O-acetyl-3-O-benzyl-α-L-idopyranosyluronate)-(1-4)-(1,6-di-O-acetyl-2-azido-3-O-benzyl-2-deoxy-α,β-D-glucopyranose) (No. 13)

Compound 12 (365 mg, 0.254 mmol) obtained in step 3.a is treated as for the synthesis of compound 9 (step 2.b) to give, after purification on silica gel (1:1 Et₂O-diisopropyl ether), 13 (376 mg, 97%).

Mass spectrum (ESI) m/z 1560.7 [(M+Na)⁺].

### Step 3.c: Preparation of (benzyl 2,4-di-O-acetyl-3-O-benzyl-α-L-idopyranosyluronate)-(1-4)-6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-O-benzyl-2-deoxy-α,β-D-glucopyranose) (No. 14)

Compound 13 (364 mg, 0.237 mmol) obtained in step 3.b is treated as for the synthesis of compound 10 (step 2.c) to give, after purification on silica gel (7:3 Et₂O-diisopropyl ether), compound 14 (310 mg, 87%). Mass spectrum (ESI) m/z 1518.8 [(M+Na)⁺].

### Step 3.d: Preparation of (benzyl 2,4-di-O-acetyl-3-O-benzyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-O-benzyl-2-deoxy-α,β-D-glucopyranose trichloroacetimidate) (No. 15)

Compound 14 (279 mg, 0.187 mmol) obtained in step 3.c is treated as for the synthesis of compound 11 (step 2.d) to give, after purification on silica gel (1:1 Et₂O-diisopropyl ether), 15 (230 mg, 75%).

Mass spectrum (ESI) m/z 1660.6 [(M+Na)⁺].

### Step 3.e: Preparation of (benzyl 2,4-di-O-acetyl-3-O-benzyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-O-benzyl-2-deoxy-α,β-D-glucopyranosyl)-(1-4)-((3S,4R,5R)-dibenzyl 5-(benzyl-oxy)-4-oxypiperidine-1,3-carboxylate) (No. 16)

Compounds 15 (217 mg, 0.132 mmol) obtained in step 3.d, and 6 (126 mg, 0.264 mmol) obtained in step 1.e, are treated according to METHOD 2 to give, after purification, compound 16 (168 mg, 66%).

Mass spectrum (ESI) m/z 1976.0 [(M+Na)⁺].

### Step 3.f: Preparation of (benzyl 2,4-di-O-acetyl-3-O-benzyl-α-L-idopyranosyluronate)-(1-4)-(6-O-acetyl-2-acetamido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-acetamido-3-O-benzyl-2-deoxy-α-D-glucopyranosyl)-(1-4)-((3S,4R,5R)-dibenzyl 5-(benzyl-oxy)-4-oxypiperidine-1,3-dicarboxylate) (No. 17)

Compound 16 (138.5 mg, 70.9 μmol) obtained in step 3.e is dissolved in pyridine (1.16 mL) and then thioacetic acid (1.14 mL, 225 molar equivalents) is added at 0° C. The reaction medium is stirred for 14 h at room temperature and is then concentrated and purified on silica gel (3:2 cyclohexane-ethyl acetate) to give compound 17 (118 mg, 84%).

Mass spectrum (ESI) m/z 2007.7 [(M+Na)⁺].

### Step 3.g: Preparation of (3-O-benzyl-α-L-idopyranosyluronic acid)-(1-4)-(2-acetamido-3-O-benzyl-2-deoxy-α-D-glucopyranosyl)-(1-4)-(3-O-benzyl-α-L-idopyranosyluronic acid)-(1-4)-(2-acetamido-3-O-benzyl-2-deoxy-α-D-glucopyranosyl)-(1-4)-((3S,4R,5R)benzyl 5-(benzyloxy)-4-oxypiperidine-3-carboxylic acid-1-carboxylate) (No. 18)

Compound 17 (101 mg, 50.9 μmol) obtained in step 3.f is treated according to METHOD 3 and then the reaction medium is acidified with 6N hydrochloric acid (pH 1) and extracted with dichloromethane. The organic phase is washed with 5% sodium sulphite (Na₂SO₃) and then with water. After drying, filtration and concentration, the residue is used in the crude state in the next step.

Mass spectrum (ESI) m/z 1505.6 [(M+H)⁺].

### Step 3.h: Preparation of (3-O-benzyl-2,4-di-O-sodium sulphonato-α-L-idopyranosyluronate of sodium)-(1-4)-(2-acetamido-3-O-benzyl-2-deoxy-6-O-sodium sulphonato-α-D-glucopyranosyl)-(1-4)-(3-O-benzyl-2-O-sodium sulphonato-α-L-idopyranosyluronate of sodium)-(1-4)-(2-acetamido-3-O-benzyl-2-deoxy-6-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 19)

The crude compound 18 obtained in step 3.g is treated according to METHOD 4 to give compound 19 (50 mg, 54% (2 steps)).

Mass spectrum (ESI) m/z 2014 [(M−3Na+3H)⁻].

### Preparation 4

### Synthesis of (3-O-benzyl-2,4-di-O-sodium sulphonato-α-L-idopyranosyluronate of sodium)-(1-4)-(3-O-benzyl-2-deoxy-2-N-sodium sulphonato-6-O-sodium sulphonato-α-D-glucopyranosyl)-(1-4)-(3-O-benzyl-2-O-sodium sulphonato-α-L-idopyranosyluronate of sodium)-(1-4)-(3-O-benzyl-2-deoxy-2-N-sodium sulphonato-6-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 26)

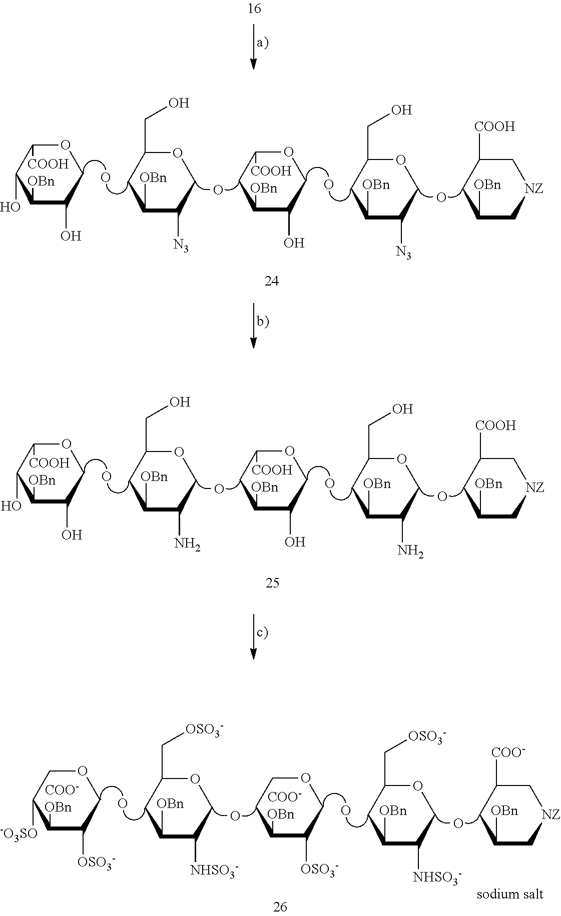

### Step 4.a: Preparation of (3-O-benzyl-α-L-idopyranosyluronic acid)-(1-4)-(2-azido-3-O-benzyl-2-deoxy-α-D-glucopyranosyl)-(1-4)-(3-O-benzyl-α-L-idopyranosyluronic acid)-(1-4)-(2-azido-3-O-benzyl-2-deoxy-α-D-glucopyranosyl)-(1-4)-(5-(benzyloxy)-4-oxypiperidine-1-carboxylate of benzyl-3-carboxylate of sodium (3S,4R,5R)) (No. 24)

Compound 16 (175 mg, 89.6 μmol) obtained in step 3.e is treated according to METHOD 3 and then the reaction medium is acidified with 6N hydrochloric acid (pH 2). The mixture is then purified on a Sephadex® LH-20 column (1:1 dichloromethane-ethanol) to give compound 24 (100 mg, 76%).

Mass spectrum (ESI) m/z 1474.1 [(M+H)⁺].

### Step 4.b: Preparation of (3-O-benzyl-α-L-idopyranosyluronic acid)-(1-4)-(2-amino-3-O-benzyl-2-deoxy-α-D-glucopyranosyl)-(1-4)-(3-O-benzyl-α-L-idopyranosyluronic acid)-(1-4)-(2-amino-3-O-benzyl-2-deoxy-α-D-glucopyranosyl)-(1-4)-(5-(benzyloxy)-4-oxypiperidine-1-carboxylate of benzyl-3-carboxylic acid (3S,4R,5R)) (No. 25)

A 10% Pd/C/ethylenediamine complex prepared according to the method described in H. Sajiki et al., J. Org. Chem. (1998), Vol. 63, p. 7990-7992) (107 mg) is added to a solution of compound 24 (35 mg) obtained in step 4.a, in a 1:1 methanol-tetrahydrofuran mixture (1 ml). The medium is then placed under a H₂ pressure (3 bar) at room temperature for 16 h. After filtration and concentration, the crude reaction product is reacted again under the same conditions and is then purified on silica gel (ethyl acetate-pyridine-acetic acid-water, 6:2:0.6:1) to give compound 25 (12 mg, 44%).

Mass spectrum (ESI) m/z 1421.4 [(M+H)⁺]

### Step 4.c: Preparation of (3-O-benzyl-2,4-di-O-sodium sulphonato-α-L-idopyranosyluronate of sodium)-(1-4)-(3-O-benzyl-2-deoxy-2-N-sodium sulphonato-6-O-sodium sulphonato-α-D-glucopyranosyl)-(1-4)-(3-O-benzyl-2-O-sodium sulphonato-α-L-idopyranosyluronate of sodium)-(1-4)-(3-O-benzyl-2-deoxy-2-N-sodium sulphonato-6-β-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 26)

Compound 25 (8.5 mg, 5.98 μmol) obtained in step 4.b is treated according to method 4 to give compound 26 (7 mg, 56%).

Mass spectrum (ESI) m/z 2133.8 [(M−H)⁻].

### Preparation 5

### Synthesis of (benzyl 2-O-acetyl-4-O-levulinoyl-3-O-methyl-α-L-idopyranosyluronate)-(1-4)-(6-O-acetyl-2-O-(3-bromo-4-methoxy)benzyl-3-O-methyl-α,β-D-gluco-pyranose trichloroacetimidate) (No. 40)

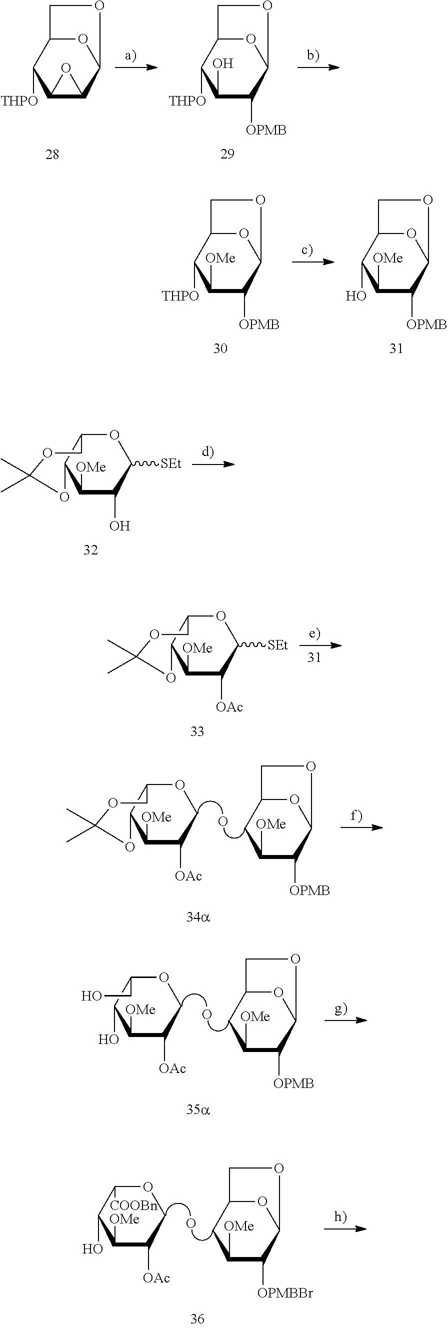

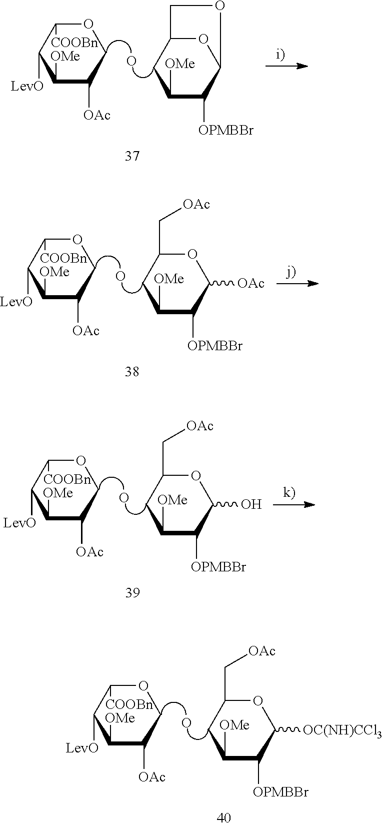

### Step 5.a: Preparation of 1,6-anhydro-4-O-(tetrahydropyran-2-yl)-2-O-(4-methoxy)benzyl-β-D-glucopyranose (No. 29)

Sodium (1.37 g, 0.33 molar equivalent) is added, at 40° C., to a solution of compound 28 (41 g, 180 mmol) (prepared according to H. Paulsen and W. Stenzel, Chem. Ber. (1978) 111, 2348-57) in para-methoxybenzyl alcohol (25 ml, 1.1 molar equivalent). The reaction mixture is then heated at 110° C. for 20 min and then methanol (20 ml) is added at 0° C. and the stirring is maintained for 16 h at room temperature. After concentration, purification on silica gel (3:7 ethyl acetate-diisopropyl ether) gives 29 (20.1 g, 31%).

Mass spectrum (ESI) m/z 384.2 [(M+Na)⁺].

### Step 5.b: Preparation of 1,6-anhydro-4-O-(tetrahydropyran-2-yl)-2-O-(4-methoxy)benzyl-3-O-methyl-β-D-glucopyranose (No. 30)

Methyl iodide (2.67 ml) and sodium hydride (2.68 g) are successively added, at 0° C. and under an argon atmosphere, to a solution of compound 29 (13.1 g, 35.8 mmol) obtained in step 5.a, in dimethylformamide (107 ml). After returning to room temperature, the reaction mixture is stirred for 16 h and then methanol is added at 0° C., the medium is extracted with ethyl acetate, dried (Na₂SO₄), filtered and concentrated to give 30 which is directly used in the next step.

Mass spectrum (ESI) m/z 403.3 [(M+Na)⁺].

### Step 5.c: Preparation of 1,6-anhydro-2-O-(4-methoxy)benzyl-3-O-methyl-β-D-glucopyranose (No. 31)

A 0.25M methanolic solution of camphor-sulphonic acid (CSA) (1 molar equivalent) is added to a solution of the crude compound 30 obtained in step 5.b, in methanol (143 ml). After 30 min of magnetic stirring, the medium is diluted with dichloromethane and then washed with water, with a 2% aqueous sodium hydrogen carbonate solution, with water, dried (Na₂SO₄), filtered and concentrated. Purification on silica gel (3:7 ethyl acetate-cyclohexane) gives compound 31 (9.0 g, 85%).

Mass spectrum (ESI) m/z 319.1 [(M+Na)⁺].

### Step 5.d: Preparation of ethyl 2-O-acetyl-4,6-β-isopropylidene-3-O-methyl-1-thio-α,β-L-idopyranoside (No. 33)

Triethylamine (2.2 ml), DMAP (173 mg) and acetic anhydride (1.34 ml) are successively added, at 0° C., under argon, to a solution of the crude compound 32 (1.99 g, 7.1 mmol) (prepared according to P. Duchaussoy et al., Carbohydr. Res. (1999), 317, 63-84) in dichloromethane (37 ml). The temperature is kept at 0° C. for 10 min and then the reaction medium is placed at room temperature for 16 h. The reaction mixture is then concentrated under vacuum and the residue purified on silica (1:1 Et₂O-cyclohexane) to give compound 33 (2.1 g, 92%).

Mass spectrum (ESI) m/z 343.3 [(M+Na)⁺].

### Step 5.e: Preparation of (2-O-acetyl-4,6-O-isoprop-ylidene-3-O-methyl-α-L-idopyranosyl)-(1-4)-(1,6-anhydro-2-O-(4-methoxy)benzyl-3-O-methyl-β-D-gluco-pyranose) (No. 34α) and (2-O-acetyl-4,6-O-isoprop-ylidene-3-O-methyl-β-L-idopyranosyl)-(1-4)-(1,6-anhydro-2-O-(4-methoxy)benzyl-3-O-methyl-β-D-gluco-pyranose) (No. 34β)

A solution of N-iodosuccinimide (1.38 g) and trifluoromethanesulphonic acid (63.5 μl) in a 1:1 dichloromethane-dioxane mixture (16.5 ml) is added, at −20° C., to a mixture, under argon, of compound 33 (1.86 g, 5.79 mmol) obtained in step 5.d, and of compound 31 (1.46 g; 4.94 mmol) obtained in step 5.c, in the presence of 4 Å molecular sieves (2.89 g) in toluene (50 ml). After stirring for 45 min, solid sodium hydrogen carbonate is added to the reaction medium, and after filtration, the mixture is diluted with dichloromethane, washed with 10% aqueous sodium thiosulphate (Na₂S₂O₃) solution and a saturated aqueous sodium chloride solution. After drying and concentrating, the residue is directly used in the next step.

Mass spectrum (ESI) m/z 577.4 [(M+Na)⁺].

### Step 5.f: Preparation of (2-O-acetyl-3-O-methyl-α-L-idopyranosyl)-(1-4)-(1,6-anhydro-2-O-(4-methoxy)benzyl-3-O-methyl-β-D-glucopyranose) (No. 35α) and (2-O-acetyl-3-O-methyl-β-L-idopyranosyl)-(1-4)-(1,6-anhydro-2-O-(4-methoxy)benzyl-3-O-methyl-β-D-glucopyranose) (No. 35β)

Acetic acid at 70% (55 ml) is added to a solution of the mixed compound 34α and 34β obtained in step 5.e, in 1,2-dichloroethane (12 ml). The mixture is heated at 60° C. for 50 min and then concentrated under vacuum and the residue obtained is purified on silica gel (3:2 toluene-acetone) to give compound 35α (1.56 g, 61%, two steps), and compound 35β (0.36 g, 14%, two steps).

Mass spectrum (ESI) m/z 537.5 [(M+Na)⁺].

### Step 5.g: Preparation of (benzyl 2-O-acetyl-3-O-methyl-α-L-idopyranosyluronate)-(1-4)-(1,6-anhydro-2-O-(3-bromo-4-methoxy)benzyl-3-O-methyl-β-D-glucopyranose) (No. 36)

Compound 35a (0.975 g, 1.89 mmol) obtained in step 5f is treated according to METHOD 1 to give, after purification on silica gel (1:1 ethyl acetate-cyclohexane), compound 36 (1.09 g, 83%).

¹H NMR (CDCl₃) δ 7.54-6.86 (m, 8H, Ar).

### Step 5.h: Preparation of (benzyl 2-O-acetyl-4-O-levulinoyl-3-O-methyl-α-L-idopyranosyluronate)-(1-4)-(1,6-anhydro-2-O-(3-bromo-4-methoxy)benzyl-3-O-methyl-β-D-glucopyranose) (No. 37)

DMAP (43 mg, 0.2 molar equivalent), 1-(3-dimethylaminopropyl)-3-ethylcarbodiimide hydrochloride (EDCl) (0.675 g, 2 molar equivalents) and levulinic acid (0.361 ml, 2 molar equivalents) are successively added to a solution, under argon, of compound 36 (1.09 g, 1.56 mmol) obtained in step 5.g, in dioxane (35 ml), and after stirring for 16 h, the reaction medium is successively washed with a 10% potassium hydrogen sulphate (KHSO₄) solution, water, 2% sodium hydrogen carbonate and then the solution is dried (Na₂SO₄), filtered and concentrated to give a residue which is purified on silica gel (ethyl acetate-dichloromethane 2:3) giving compound 37 (1.07 g, 86%). Mass spectrum (ESI) m/z 819.4 [(M+Na)⁺].

### Step 5.i: Preparation of (benzyl 2-O-acetyl-4-O-levulinoyl-3-O-methyl-α-L-idopyranosyluronate)-(1-4)-(1,6-di-O-acetyl-2-O-(3-bromo-4-methoxy)benzyl-3-β-methyl-α,β-D-glucopyranose) (No. 38)

Compound 37 (1.07 g, 1.34 mmol) obtained in step 5.h is treated as for the synthesis of compound 9 (step 2.b) to give, after purification on silica gel (ethyl acetate-dichloromethane 2:3), compound 38 (1.04 g, 87%).

Mass spectrum (ESI) m/z 921.4 [(M+Na)⁺].

### Step 5.j: Preparation of (benzyl 2-O-acetyl-4-O-levulinoyl-3-O-methyl-α-L-idopyranosyluronate)-(1-4)-(6-O-acetyl-2-O-(3-bromo-4-methoxy)benzyl-3-O-methyl-α,β-D-glucopyranose) (No. 39)

Compound 38 (1.04 g, 1.16 mmol) obtained in step 5.i is treated as for the synthesis of compound 10 (step 2.c) to give, after purification on silica gel (ethyl acetate-dichloromethane 1:1), compound 39 (740 mg, 74%).

Mass spectrum (ESI) m/z 877.3 [(M+Na)⁺].

### Step 5.k: Preparation of (benzyl 2-O-acetyl-4-O-levulinoyl-3-O-methyl-α-L-idopyranosyluronate)-(1-4)-(6-O-acetyl-2-O-(3-bromo-4-methoxy)benzyl-3-O-methyl-α,β-D-glucopyranose trichloroacetimidate) (No. 40)

Compound 39 (740 mg, 860 μmol) obtained in step 5.j is treated as for the synthesis of compound 11 to give, after purification on silica gel (ethyl acetate-dichloromethane 3:7), compound 40 (714 mg, 83%).

¹H NMR (CDCl₃) δ 6.39 (d, H−1α Glc^{I}), 5.76 (d, H−1β Glc^{I}).

### Preparation 6

### Synthesis of (3-O-methyl-2,4-di-O-sodium sulphonato-α-L-idopyranosyluronate of sodium)-(1-4)-(3-O-methyl-2,6-di-O-sodium sulphonato-α-D-glucopyranosyl)-(1-4)-(3-O-methyl-2-O-sodium sulphonato-α-L-idopyranosyluronate of sodium)-(1-4)-(3-O-methyl-2,6-di-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (compound No. 46)

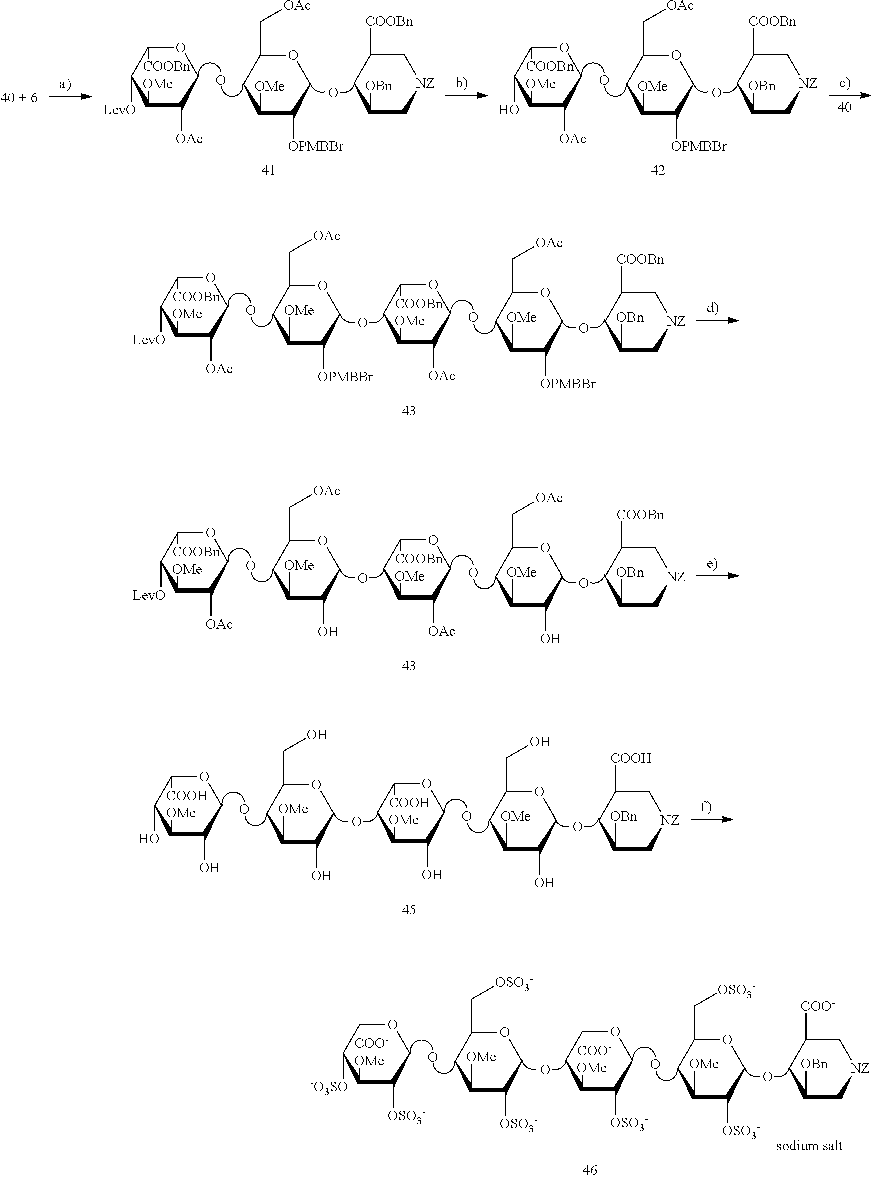

### Step 6.a: Preparation of (benzyl 2-O-acetyl-4-O-levulinoyl-3-O-methyl-α-L-idopyranosyluronate)-(1-4)-(6-O-acetyl-2-O-(3-bromo-4-methoxy)benzyl-3-O-methyl-α-5-(benzyl-oxy)-4-oxypiperidine-1,3-dicarboxylate) (No. 41)

A mixture of compound 40 (94 mg, 0.094 mmol, 1.0 molar equivalent) obtained in step 5.k, and of compound 6 (111 mg, 0.234 mmol, 2.5 molar equivalents) obtained in step 1.e, is treated according to METHOD 2 to give, after purification on Sephadex® LH-20 column, and then on silica gel (dichloromethane-ethyl acetate 9:1), compound 41 (62 mg, 50%).

Mass spectrum (ESI) m/z 1336.5 [(M+Na)⁺].

### Step 6.b: Preparation of (benzyl 2-O-acetyl-3-O-methyl-α-L-idopyranosyluronate)-(1-4)-(6-O-acetyl-2-O-(3-bromo-4-methoxy)benzyl-3-O-methyl-α-D-glucopyranosyl)-(1-4)-((3S,4R,5R)-dibenzyl 5-(benzyloxy)-4-oxypiperidine-1,3-dicarboxylate) (No. 42)

Hydrazine acetate (21 mg, 10 molar equivalents) is added to a solution of compound 41 (60 mg, 45.7 μmol) obtained in step 6.a, in a 1:2 toluene/ethanol mixture (9 ml). After 3 h of magnetic stirring, the mixture is concentrated under vacuum and the residue is diluted with dichloromethane, washed with a 2% sodium hydrogen carbonate solution, with water, and the organic phase is dried (Na₂SO₄), filtered and then concentrated. The residue is purified on silica gel (ethyl acetate-cyclohexane 3:2) to give compound 42 (48 mg, 88%).

Mass spectrum (ESI) m/z 1238.5 [(M+Na)⁺].

### Step 6.c: Preparation of (benzyl 2-O-acetyl-4-O-levulinoyl-3-O-methyl-α-L-idopyranosyluronate)-(1-4)-(6-O-acetyl-2-O-(3-bromo-4-methoxy)benzyl-3-O-methyl-α-D-glucopyranosyl)-(1-4)-(benzyl 2-O-acetyl-3-O-methyl-α-L-idopyranosyluronate)-(1-4)-(6-O-acetyl-2-O-(3-bromo-4-methoxy)benzyl-3-O-methyl-α-D-glucopyranosyl)-(1-4)-((3S,4R,5R)-dibenzyl 5-(benzyloxy)-4-oxypiperidine-1,3-dicarboxylate) (No. 43)

A mixture of compound 40 (197.7 mg, 197.7 μmol, 1.14 molar equivalents) obtained in step 5.k, and of compound 42 (210.5 mg, 173.2 μmol, 1.0 molequiv.) obtained in step 6.b, is treated according to method 2 to give, after purification on a Sephadex® LH-20 column, and then on silica gel (toluene-acetone 1:1), compound 43 (176 mg, 50%). Mass spectrum (ESI) m/z 2075.0 [(M+Na)⁺].

### Step 6.d: Preparation of (benzyl 2-O-acetyl-4-O-levulinoyl-3-O-methyl-α-L-idopyranosyluronate)-(1-4)-(6-O-acetyl-3-O-methyl-α-D-glucopyranosyl)-(1-4)-(benzyl 2-O-acetyl-3-O-methyl-α-L-idopyranosyluronate)-(1-4)-(6-O-acetyl-3-O-methyl-α-D-glucopyranosyl)-(1-4)-((3S,4R,5R)-dibenzyl 5-(benzyloxy)-4-oxypiperidine-1,3-dicarboxylate) (No. 44)

Zirconium tetrachloride (ZrCl₄) (39 mg, 5 molar equivalents) is added, at 0° C. and under an argon atmosphere, to a solution of compound 43 (69 mg, 33.6 μmol) obtained in step 6.c, in acetonitrile (3 ml). After stirring for 45 min at room temperature, the mixture is concentrated under vacuum and the residue is diluted with ethyl acetate, washed with water, and after drying, filtration and concentration, the residue is purified on silica gel (3:7 acetone-toluene) to give compound 44 (52 mg, 89%).

Mass spectrum (ESI) m/z 1676.7 [(M+Na)⁺].

### Step 6.e: Preparation of (3-O-methyl-α-L-idopyranosyluronic acid)-(1-4)-(3-O-methyl-α-D-glucopyranosyl)-(1-4)-(3-O-methyl-α-L-idopyranosyluronic acid)-(1-4)-(3-O-methyl-α-D-glucopyranosyl)-(1-4)-(5-(benzyloxy)-4-oxypiperidine-1-carboxylate of benzyl-3-carboxylic acid (3S,4R,5R)) (No. 45)

Compound 44 (18 mg, 11 μmol) obtained in step 6.d is treated according to METHOD 3 to give, after purification, compound 45 (5.8 mg, 47%) which may be partially esterified on the carboxylic acid groups.

Mass spectrum (ESI) m/z 1118.4 [(M+H)⁺].

### Step 6.f: Preparation of (3-O-methyl-2,4-di-O-sodium sulphonato-α-L-idopyranosyluronate of sodium)-(1-4)-(3-O-methyl-2,6-di-O-sodium sulphonato-α-D-gluco-pyranosyl)-(1-4)-(3-O-methyl-2-O-sodium sulphonato-α-L-idopyranosyluronate of sodium)-(1-4)-(3-O-methyl-2,6-di-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 46)

Compound 45 (7 mg, 6.26 μmol) obtained in step 6.e is treated according to METHOD 4 to give, after purification, compound 46 (10 mg, 77%) which may be partially esterified on the carboxylic acid groups.

Mass spectrum (ESI) m/z 1897.0 [(M+Na−H)⁺].

### Preparation 7

### Synthesis of (benzyl 2-O-acetyl-4-O-levulinoyl-3-β-benzyl-α-L-idopyranosyluronate)-(1-4)-(6-O-acetyl-2-β-(4-methoxy)benzyl-3-O-benzyl-α,β-D-glucopyranose trichloroacetimidate) (No. 57)

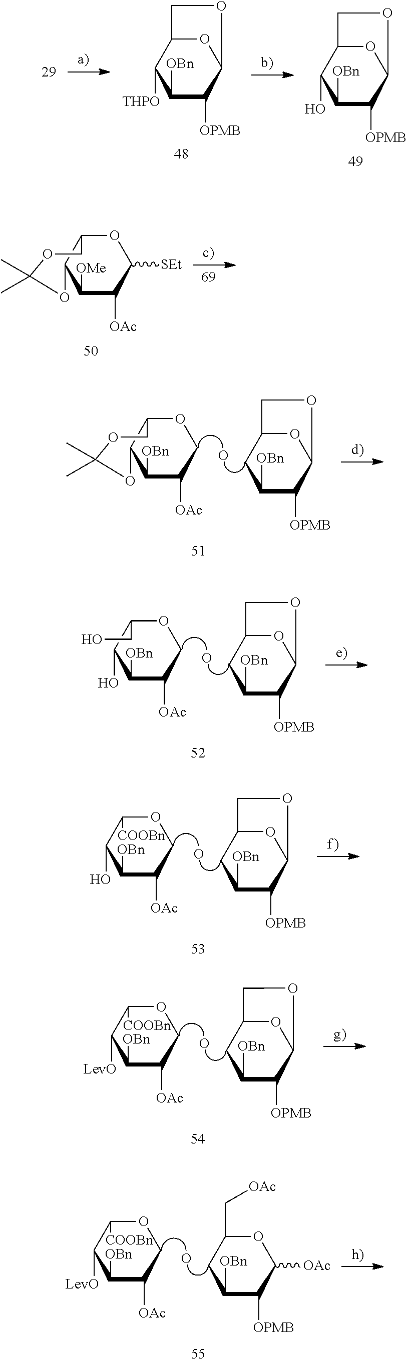

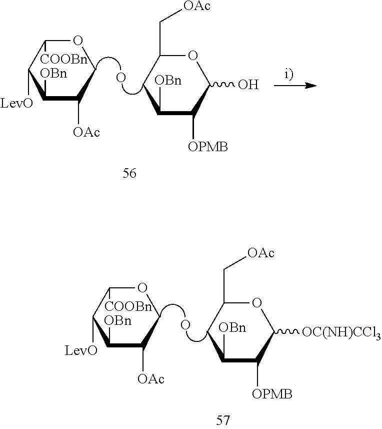

### Step 7.a: Preparation of 1,6-anhydro-4-O-(tetrahydropyran-2-yl)-2-O-(4-methoxy)benzyl-3-O-benzyl-β-D-glucopyranose (No. 48)

Benzyl bromide (5.1 ml) and then sodium hydride (4.6 g) are added, at 0° C., to a solution of 29 (19.96 g, 54.5 mmol) obtained in step 5.a, in DMF (300 ml). At the end of the addition, the mixture is placed at room temperature for 16 h and then methanol (18 ml) is added at 0° C., and after stirring for 1 h at room temperature, the medium is diluted with ethyl acetate (600 ml), washed with water (300 ml), dried (Na₂SO₄), filtered and concentrated. The residue is purified by flash chromatography (5:95 ethyl acetate-diisopropyl ether) to give 48 (20.6 g, 83%).

Mass spectrum (ESI) m/z 479.3 [(M+Na)⁺].

### Step 7.b: Preparation of 1,6-anhydro-2-O-(4-methoxy)-benzyl-3-O-benzyl-β-D-glucopyranose (No. 49)

Compound 48 (20.6 g, 45.2 mmol) obtained in step 7.a is treated as for the synthesis of compound 31 (step 5.c) to give, after purification on silica (3:7 ethyl acetate-cyclohexane), 49 (14.4 g, 86%).

Mass spectrum (ESI) m/z 395.4 [(M+Na)⁺].

### Step 7.c: Preparation of (2-O-acetyl-4,6-O-isopropylidene-3-O-benzyl-α,β-L-idopyranosyl)-(1-4)-(1,6-anhydro-2-O-(4-methoxy)benzyl-3-O-benzyl-β-D-glucopyranose) (No. 51)

Compound 50 (prepared according to the method described by C. Tabeur et al. for the 2-O-benzoylated derivative, Carbohydr. Res. (1996), 281, 253-276) (16.91 g, 42.6 mmol) and compound 49 (14.44 g, 38.8 mmol) obtained in step 7.b are reacted as for the synthesis of 34 (step 5.e) to give, after purification on silica (15:85 ethyl acetate-diisopropyl ether), compound 51 (17.05 g, 62% (56% alpha-L)).

Mass spectrum (ESI) m/z 729.3 [(M+Na)⁺].

### Step 7.d: Preparation of (2-O-acetyl-3-O-benzyl-α-L-idopyranosyl)-(1-4)-(1,6-anhydro-2-O-(4-methoxy)benzyl-3-O-benzyl-β-D-glucopyranose) (No. 52)

Compound 51 (12.38, 17.51 mmol) obtained in step 5.c is treated as for the synthesis of 35 (step 5.f) to give, after purification on silica (4:1 toluene-acetone), 52 (10.85 g, 93%).

Mass spectrum (ESI) m/z 689.3 [(M+Na)⁺].

### Step 7.e: Preparation of (benzyl 2-O-acetyl-3-O-benzyl-α-L-idopyranosyluronate)-(1-4)-(1,6-anhydro-2-O-(4-methoxy)benzyl-3-O-benzyl-β-D-glucopyranose) (No. 53)

Compound 52 (10.85 g, 16.27 mmol) obtained in step 7.d is treated according to METHOD 1 to give, after purification on silica (3:7 acetone-cyclohexane), 53 (8.44 g, 61%).

Mass spectrum (ESI) m/z 793.3 [(M+Na)⁺].

### Step 7.f: Preparation of (benzyl 2-O-acetyl-3-O-benzyl-4-O-levulinoyl-α-L-idopyranosyluronate)-(1-4)-(1,6-anhydro-2-O-(4-methoxy)benzyl-3-O-benzyl-β-D-glucopyranose) (No. 54)

Compound 53 (3.2 g, 3.77 mmol) obtained in step 7.e is treated as for the synthesis of 37 (step 5.h) to give 54 (3.17 g, 89%) after purification on silica (acetone-toluene 1:4).

Mass spectrum (ESI) m/z 891.3 [(M+Na)⁺].

### Step 7.g: Preparation of (benzyl 2-O-acetyl-4-O-levulinoyl-3-O-benzyl-α-L-idopyranosyluronate)-(1-4)-(1,6-di-O-acetyl-4-O-(4-methoxy)benzyl-3-O-benzyl-α,β-D-glucopyranose) (No. 55)

Compound 54 (1.45 g, 1.53 mmol) obtained in step 7.f is treated as for the synthesis of 9 but at 0° C. for 1 h to give, after purification on silica (toluene-acetone 85:15), 55 (1.25 g, 78%).

Mass spectrum (ESI) m/z 993.3 [(M+Na)⁺].

### Step 7.h: Synthesis of (benzyl 2-O-acetyl-4-O-levulinoyl-3-O-benzyl-α-L-idopyranosyluronate)-(1-4)-(6-O-acetyl-2-O-(4-methoxy)benzyl-3-O-benzyl-α,β-D-glucopyranose) (No. 56)

Compound 55 obtained in step 7.g is treated as for the synthesis of 10 (step 2.c) to give, after purification on silica (diethyl ether-dichloromethane 25:75), 56 (429 mg, 56%).

Mass spectrum (ESI) m/z 951.3 [(M+Na)⁺].

### Step 7.i: Preparation of (benzyl 2-O-acetyl-4-O-levulinoyl-3-O-benzyl-α-L-idopyranosyluronate)-(1-4)-(6-O-acetyl-2-O-(4-methoxy)benzyl-3-O-benzyl-α,β-D-glucopyranose trichloroacetimidate) (No. 57)

Compound 56 (429 mg, 426 μmol) obtained in step 7.h is treated as for the synthesis of 11 (step 2.d) to give, after purification on silica (ethyl acetate-cyclohexane 1:1), the derivative 57 (396 mg, 80%).

¹H NMR (CDCl₃) δ 6.45. (d, H−1α), 5.89 (d, H−1β).

### Preparation 8

### Preparation of (benzyl 2-O-acetyl-4-O-allyl-3-O-benzyl-α-L-idopyranosyluronate)-(1-4)-(6-O-acetyl-2-O-(4-methoxy)benzyl-3-O-benzyl-α,β-D-glucopyranose trichloroacetimidate) (No. 62)

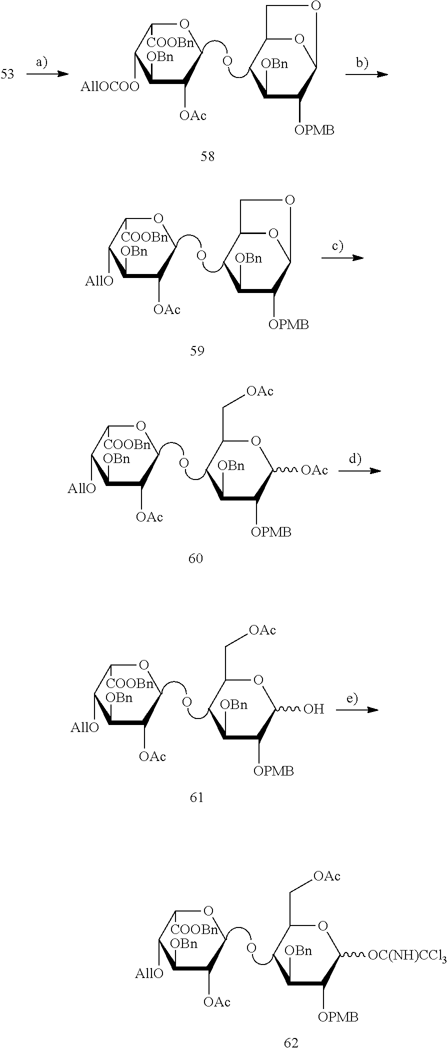

### Step 8.a: Preparation of (benzyl 2-O-acetyl-4-O-allyloxycarbonyl-3-O-benzyl-α-L-idopyranosyluronate)-(1-4)-(1,6-anhydro-2-O-(4-methoxy)benzyl-3-O-benzyl-β-D-glucopyranose)(No. 58)

Pyridine (759 μl, 9.41 mmol), DMAP (115 mg, 0.94 mmol) and allyl chloroformate (995 μl, 9.41 mmol) in solution in THF (2.35 ml) are added, at 0° C. and under an argon atmosphere, to a solution of 53 (0.800 g, 0.9414 mmol) obtained in step 7.e, in THF (9.4 ml). The stirring is maintained overnight and then the reaction mixture is diluted with ethyl acetate, washed with 10% KHSO₄, with 2% sodium hydrogen carbonate, with water, dried (Na₂SO₄), filtered and concentrated. The residue is purified by flash chromatography (1:9 acetone-toluene), to give 58 (0.809 g, 92%).

Mass spectrum (ESI) m/z 877.3 [(M+Na)⁺].

### Step 8.b: Preparation of (benzyl 2-O-acetyl-4-O-allyl-3-O-benzyl-α-L-idopyranosyluronate)-(1-4)-(1,6-anhydro-2-O-(4-methoxy)benzyl-3-O-benzyl-β-D-glucopyranose) (No. 59)

Palladium diacetate (3.9 mg, 0.017 mmol) and triphenylphosphine (22.6 mg, 0.086 mmol) are successively added, under an argon atmosphere, to a solution, in THF (6 ml), of compound 58 (0.805 g, 0.862 mmol) obtained in step 8.a. The temperature of the mixture is brought to 90° C. for 15 min and then the medium is concentrated under vacuum and purified on silica (15:85 acetone-toluene) to give 59 (0.587 g, 66%).

Mass spectrum (ESI) m/z 833.4 [(M+Na)⁺].

### Step 8.c: Preparation of (benzyl 2-O-acetyl-4-O-allyl-3-O-benzyl-α-L-idopyranosyluronate)-(1-4)-(1,6-di-O-acetyl-2-O-(4-methoxy)benzyl-3-O-benzyl-α,β-D-glucopyranose) (No. 60)

Compound 59 (0.587 g, 0.660 mmol) obtained in step 8.b is treated as for the synthesis of compound 55 (step 7.g) to give, after purification on silica (9:1 acetone-toluene), 60 (0.37 g, 57%).

Mass spectrum (ESI) m/z 936.4 [(M+Na)⁺].

### Step 8.d: Preparation of (benzyl 2-O-acetyl-4-O-allyl-3-O-benzyl-α-L-idopyranosyluronate)-(1-4)-(6-O-acetyl-2-O-(4-methoxy)benzyl-3-O-benzyl-α,β-D-glucopyranose) (No. 61)

Compound 60 obtained in step 8.c is treated as for the synthesis of 10 (step 2.c) to give, after purification on silica gel (cyclohexane-ethyl acetate 2:3), 61 (240 mg, 70%).

Mass spectrum (ESI) m/z 893.4 [(M+Na)⁺].

### Step 8.e: Preparation of (benzyl 2-O-acetyl-4-allyl-3-O-benzyl-α-L-idopyranosyluronate)-(1-4)-(6-O-acetyl-2-O-(4-methoxy)benzyl-3-O-benzyl-α,β-D-glucopyranose trichloroacetimidate) (No. 62)

Compound 61 (234 mg, 246 μmol) obtained in step 8.d is treated as for the synthesis of 11 (step 2.d) to give, after purification on silica (86:14 acetone-toluene), the derivative 62 (249 mg, 93%).

¹H NMR (CDCl₃) δ 6.40 (d, H−1α), 5.81 (d, H−1β).

The syntheses of PREPARATIONS 9 and 10 may be schematically represented as follows:

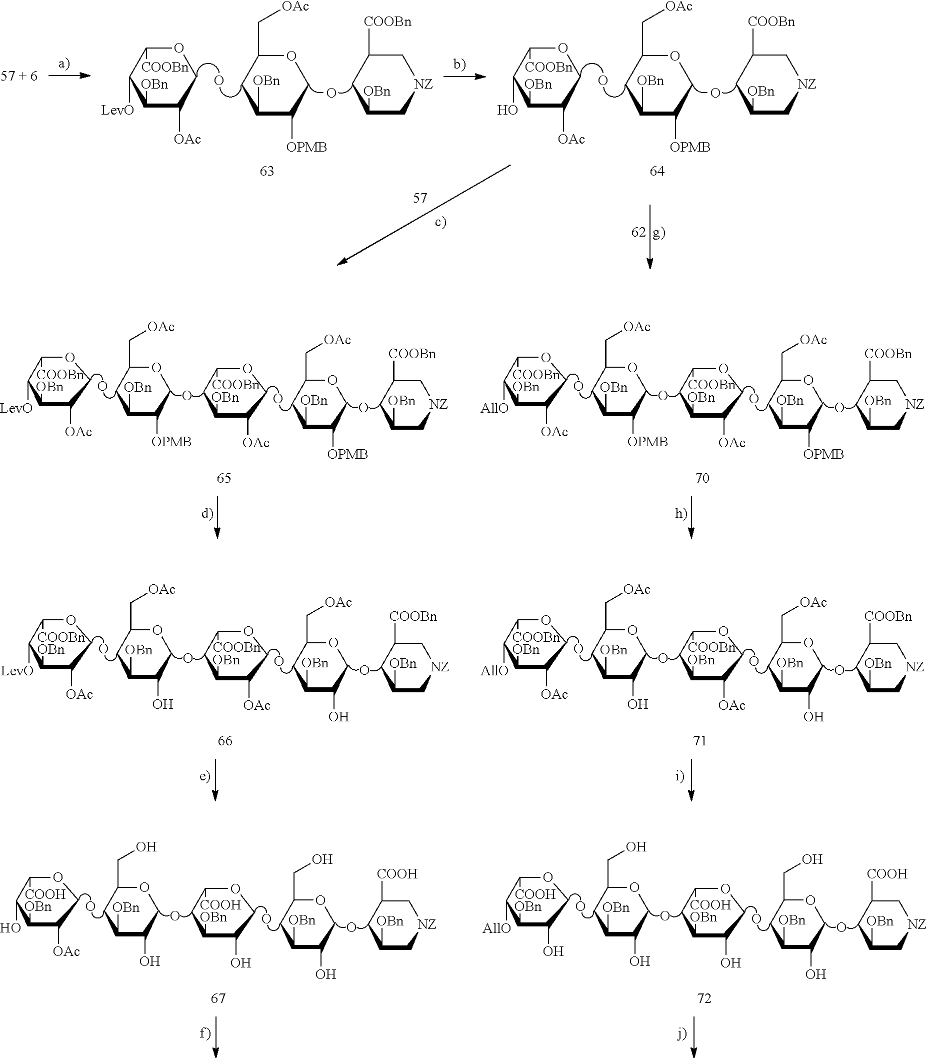

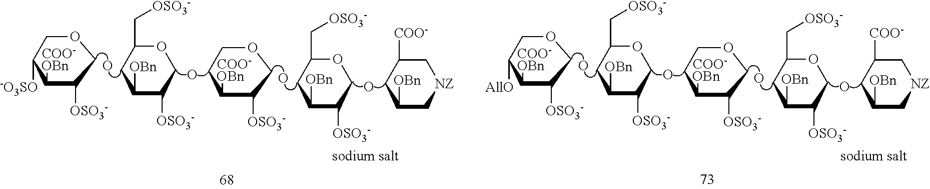

### Preparation 9

### Synthesis of (3-O-benzyl-2,4-di-O-sodium sulphonato-α-L-idopyranosyluronate of sodium)-(1-4)-(3-O-benzyl-2,6-di-O-sodium sulphonato-α-D-glucopyranosyl)-(1-4)-(3-O-benzyl-2-O-sodium sulphonato-α-L-idopyranosyluronate of sodium)-(1-4)-(3-O-benzyl-2,6-di-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 68)

### Step 9.a: Preparation of (benzyl 2-O-acetyl-4-O-levulinoyl-3-O-benzyl-α-L-idopyranosyluronate)-(1-4)-(6-O-acetyl-2-O-(4-methoxy)benzyl-3-O-benzyl-α,β-D-glucopyranosyl)-(1-4)-((3S,4R,5R)-dibenzyl 5-(benzyloxy)-4-oxypiperidine-1,3-dicarboxylate) (No. 63)

Compound 57 (396 mg, 0.34 mmol) obtained in step 7.i, and compound 6 (405 mg, 0.85 mmol) obtained in step 1.e, are treated according to METHOD 2 to give, after purification, 63 (369 mg, 73%).

¹H NMR (CDCl₃) δ 5.29 (d, H−1 Glc^{II}), 5.13 (d, H−1 IdoUA^{III}).

### Step 9.b: Preparation of (benzyl 2-O-acetyl-3-O-benzyl-α-L-idopyranosyluronate)-(1-4)-(6-O-acetyl-2-O-(4-methoxy)benzyl-3-O-benzyl-α-D-glucopyranosyl)-(1-4)-((3S,4R,5R)-dibenzyl 5-(benzyloxy)-4-oxypiperidine-1,3-dicarboxylate) (No. 64)

Compound 63 (372 mg, 0.254 mmol) obtained in step 9.a is treated as for the synthesis of 42 (step 6.b) to give, after purification on silica (ethyl acetate-cyclohexane 2:3), compound 64 (301 mg, 87%).

¹H NMR (CDCl₃) δ 5.29 (d, H−1 Glc^{II}), 5.10 (d, H−1 IdoUA^{III}), 3.97 (dd, H−4 IdoUA^{III}).

### Step 9.c: Preparation of (benzyl 2-O-acetyl-4-O-levulinoyl-3-O-benzyl-α-L-idopyranosyluronate)-(1-4)-(6-O-acetyl-2-O-(4-methoxy)benzyl-3-O-benzyl-α-D-glucopyranosyl)-(1-4)-(benzyl 2-O-acetyl-3-O-benzyl-α-L-idopyranosyluronate)-(1-4)-(6-O-acetyl-2-O-(4-methoxy)benzyl-3-O-benzyl-α-D-glucopyranosyl)-(1-4)-((3S,4R,5R)-dibenzyl 5-(benzyloxy)-4-oxypiperidine-1,3-dicarboxylate) (No. 65)

Compounds 57 (124 mg, 0.108 mmol) obtained in step 7.i, and 64 (150 mg, 0.110 mmol) obtained in step 9.b, are treated according to METHOD 2 to give, after purification, 65 (90 mg, 35%).

¹H NMR (CDCl₃) δ 5.28 (d, H−1 Glc^{II}), 5.16 (d, H−1 IdoUA^{III}), 5.13 (d, H−1 IdoUA^{V}), 4.74 (d, H−1 Glc^{IV}).

### Step 9.d: Preparation of (benzyl 2-O-acetyl-4-O-levulinoyl-3-O-benzyl-α-L-idopyranosyluronate)-(1-4)-(6-O-acetyl-3-O-benzyl-α-D-glucopyranosyl)-(1-4)-(benzyl 2-O-acetyl-3-O-benzyl-α-L-idopyranosyluronate)-(1-4)-(6-O-acetyl-3-O-benzyl-α-D-glucopyranosyl)-(1-4)-((3S,4R,5R)-dibenzyl 5-(benzyloxy)-4-oxypiperidine-1,3-dicarboxylate) (No. 66)

Compound 65 (45 mg, 0.19 mmol) obtained in step 9.c is treated as for the synthesis of 44 (step 6.d) to give, after purification on silica (ethyl acetate-cyclohexane 3:2), compound 66 (34 mg, 91%).

Mass spectrum (ESI) m/z 1982.0 [(M+Na)⁺].

### Step 9.e: Preparation of (3-O-benzyl-α-L-idopyranosyl-uronic acid)-(1-4)-(3-O-benzyl-α-D-glucopyranosyl)-(1-4)-(3-O-benzyl-α-L-idopyranosyluronic acid)-(1-4)-(3-O-benzyl-α-D-glucopyranosyl)-(1-4)-(5-(benzyloxy)-4-oxypiperidine-1-carboxylate of benzyl-3-carboxylic acid (3S,4R,5R)) (No. 67)

Compound 66 (25 mg, 12.8 μmol) obtained in step 9.d is treated according to METHOD 3. The reaction mixture is acidified with 6N hydrochloric acid (pH 2) and then deposited on an LH-20 column (100 ml) equilibrated in a 9:1 DMF/water mixture. The fractions containing the product are then concentrated and purified on silica (dichloromethane-methanol 7:3) to give 67 (10.4 mg, 59%) which may be partially esterified on the carboxylic acid groups.

Mass spectrum (ESI) m/z 1422.7 [(M+H)⁺].

### Step 9.f: Preparation of (3-O-benzyl-2,4-di-O-sodium sulphonato-α-L-idopyranosyluronate of sodium)-(1-4)-(3-O-benzyl-2,6-di-O-sodium sulphonato-α-D-gluco-pyranosyl)-(1-4)-(3-O-benzyl-2-O-sodium sulphonato-α-L-idopyranosyluronate of sodium)-(1-4)-(3-O-benzyl-2,6-di-O-sodium sulphonato-α-D-glucopyranosyl)-(1-4)-(5-(benzyloxy)-4-oxypiperidine-1-carboxylate of benzyl-3-carboxylate of sodium (3S,4R,5R))(No. 68)

Compound 67 (10.4 mg, 9 μmol) obtained in step 9.e is treated according to METHOD 4 to give 68 which is used directly in the next step.

### Preparation 10

### Synthesis of (4-O-allyl-3-O-benzyl-2-O-sodium sulphonato-α-L-idopyranosyluronate of sodium)-(1-4)-(3-O-benzyl-2,6-di-O-sodium sulphonato-α-D-glucopyranosyl)-(1-4)-(3-O-benzyl-2-O-sodium sulphonato-α-L-idopyranosyluronate of sodium)-(1-4)-(3-O-benzyl-2,6-di-O-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 73)

### Step 10.g: Preparation of (benzyl 2-O-acetyl-4-O-allyl-3-O-benzyl-α-L-idopyranosyluronate)-(1-4)-(6-O-acetyl-2-O-(4-methoxy)benzyl-3-O-benzyl-α-D-glucopyranosyl)-(1-4)-(benzyl 2-O-acetyl-3-O-benzyl-α-L-idopyranosyl-uronate)-(1-4)-(6-O-acetyl-2-O-(4-methoxy)benzyl-3-O-benzyl-α-D-glucopyranosyl)-(1-4)-((3S,4R,5R)-dibenzyl-5-(benzyloxy)-4-oxypiperidine-1,3-dicarboxylate) (No. 70)

Compound 62 (244 mg, 0.223 mmol) obtained in step 8.e and compound 64 (138 mg, 0.101 mmol) obtained in step 9.b′ are treated according to METHOD 2 to give, after purification, 70 (100 mg, 43%).

¹H NMR (CDCl₃) δ 5.28 (d, H−1 Glc^{II}), 5.25 (d, H−1 IdoUA^{V}), 5.16 (d, H−1 IdoUA^{III}), 4.72 (d, H−1 Glc^{IV}).

### Step 10.h: Preparation of (benzyl 2-O-acetyl-4-O-allyl-3-O-benzyl-α-L-idopyranosyluronate)-(1-4)-(6-O-acetyl-3-O-benzyl-α-D-glucopyranosyl)-(1-4)-(benzyl 2-O-acetyl-3-O-benzyl-α-L-idopyranosyluronate)-(1-4)-(6-O-acetyl-3-O-benzyl-α-D-glucopyranosyl)-(1-4)-((3S,4R,5R)-dibenzyl 5-(benzyloxy)-4-oxypiperidine-1,3-dicarboxylate)(No. 71).

Compound 70 (92 mg, 40.0 μmol) obtained in step 10.g is treated as for the synthesis of compound 44 (step 6.d) to give, after purification on silica (acetone-toluene 17:83), compound 71 (44 mg, 59%). Mass spectrum (ESI) m/z 1924.0 [(M+Na)⁺].

### Step 10.i: Preparation of (4-O-allyl-3-O-benzyl-α-L-idopyranosyluronic acid)-(1-4)-(3-O-benzyl-α-D-glucopyranosyl)-(1-4)-(3-O-benzyl-α-L-idopyranosyl-uronic acid)-(1-4)-(3-O-benzyl-α-D-glucopyranosyl)-(1-4)-(5-(benzyloxy)-4-oxypiperidine-1-carboxylate of benzyl-3-carboxylic acid (3S,4R,5R)) (No. 72)

Compound 71 (41 mg, 21.6 μmol) obtained in step 10.h is treated according to method 3. The reaction mixture is deposited on an LH-20 column (210 ml) equilibrated in a 1:1 dichloromethane-ethanol mixture. The fractions containing the product are then concentrated and purified on silica to give 72 (30.3 mg, 96%) which may be partially esterified on the carboxylic acid groups.

Mass spectrum (ESI) m/z 1462.4 [(M+H)⁺].

### Step 10.j: Preparation of (4-O-allyl-3-O-benzyl-2-sodium sulphonato-α-L-idopyranosyluronate of sodium)-(1-4)-(3-O-benzyl-2,6-di-O-sodium sulphonato-α-D-gluco-pyranosyl)-(1-4)-(3-O-benzyl-2-O-sodium sulphonato-α-L-idopyranosyluronate of sodium)-(1-4)-(3-O-benzyl-2,6-di-O-sodium sulphonato-α-D-glucopyranosyl)-(1-4)-(5-(benzyloxy)-4-oxypiperidine-1-carboxylate of benzyl-3-carboxylate of sodium (3S,4R,5R))(No. 73).

Compound 72 (10.0 mg, 6.44 μmol) obtained in step 10.i is treated according to method 4 to give 73 which is used directly in the next step.

### Preparation 11

### Synthesis of methyl (2-N-sodium sulphonato-2,4-dideoxy-4-formyl-3,6-di-O-sodium sulphonato-α-D-gluco-pyranosyl)-(1-4)-(sodium 2-O-sodium sulphonato-α-L-idopyranosyluronate)-(1-4)-(2-N-sodium sulphonato-2-deoxy-6-O-sodium sulphonato-α-D-glucopyranoside) (No. 89)

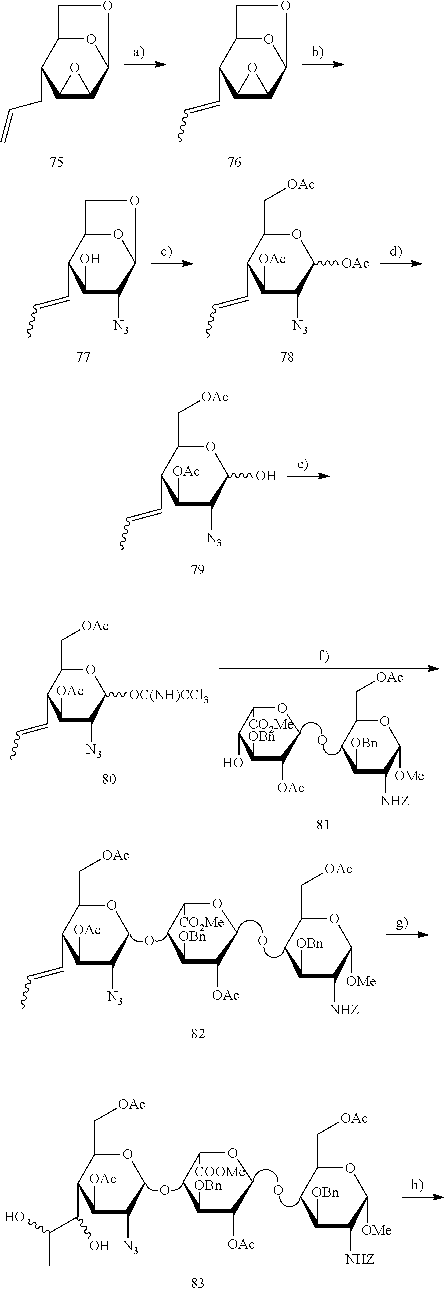

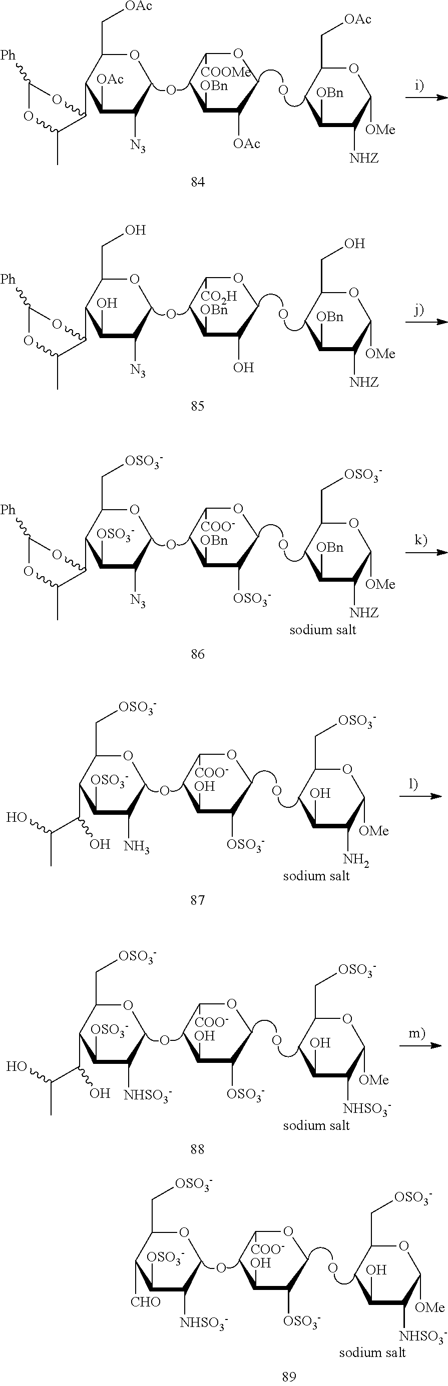

### Step 11.a: Preparation of 1,6:2,3-di-anhydro-4-deoxy-4-(prop-1-en-1-yl)-β-D-mannopyranose (No. 76)

Rhodium trichloride monohydrate (202 mg, 0.15 molar equivalent) is added, under argon, to a solution of epoxide 75 (1.2 g, 7.14 mmol) (prepared according to A G Kelly and J S Roberts, J. Chem. Soc., Chem. Commun., (1980), Vol 288) in ethanol (56.5 ml). After stirring for 1 h 25 min at 75° C., the reaction medium is poured over 250 ml of ice-cold water, and then after stirring for 5 min, the product is extracted with diethyl ether, dried (Na₂SO₄) and concentrated. The residue is then purified on silica (diisopropyl ether-cyclohexane 45:55) and the fractions containing compound 76 are partially concentrated (76 is volatile).

¹H NMR (CDCl₃) δ 3.42 (dd, H−2), 3.00 (dd, H−3), 2.64 (dd, H−4).

### Step 11.b: Preparation of 1,6-anhydro-2-azido-2,4-dideoxy-4-(prop-1-en-1-yl)-β-D-glucopyranose (No. 77)

Compound 76 obtained in step 11.a is dissolved in a dimethylformamide-water mixture (40 ml, 4:1) then sodium azide (7.0 g) is added, and the mixture is heated under reflux for 10.5 h. The reaction medium is then extracted with ethyl acetate, washed with water and then with a saturated aqueous sodium chloride solution, dried (Na₂SO₄), concentrated and purified on silica gel to give compound 77 (674 mg, 48%).

¹H NMR (CDCl₃) δ 5.8-5.6 (m, 2H, CH═CH).

### Step 11.c: Preparation of 1,3,6-tri-O-acetyl-2-azido-2,4-dideoxy-4-(prop-1-en-1-yl)-α,β-D-glucopyranose (No. 78)

Compound 77 (3.5 g, 16.57 mmol) obtained in step 11.b is treated as for the synthesis of compound 9 (step 2.b) to give, after purification, compound 78 (5.88 g, 100%).

Mass spectrum (ESI) m/z 378 [(M+Na)⁺].

### Step 11.d: Preparation of 3,6-di-O-acetyl-2-azido-2,4-dideoxy-4-(prop-1-en-1-yl)-α,β-D-glucopyranose (No. 79)

Ethanolamine (4.0 ml, 4 molar equivalents) is added, at 0° C., to a solution of compound 78 (5.88 g, 16.5 mmol) obtained in step 11.c, in tetrahydrofuran (140 ml). After 16 h at +4° C., the medium is diluted with ethyl acetate, acidified (1N HCl), washed with water, dried (Na₂SO₄) and concentrated to give, after purification, compound 79 (4.66 g, 90%).

Mass spectrum (ESI) m/z 336 [(M+Na)⁺].

### Step 11.e: Preparation of 3,6-di-O-acetyl-2-azido-2,4-dideoxy-4-(prop-1-en-1-yl)-α,β-D-glucopyranose trichloroacetimidate (No. 80)

Potassium carbonate (K₂CO₃) (3.34 g, 1.6 molar equivalents) and then CCl₃CN (7.6 ml, 5 molar equivalents) are added, under argon, to a solution of compound 79 (4.66 g, 14.9 mmol) obtained in step 11.d, in dichloromethane (285 ml). After 17 h of magnetic stirring, the reaction mixture is filtered, concentrated and purified on silica to give compound 80 (5.65 g, 83%).

¹H NMR (CDCl₃) δ 8.77 (s, NH (isomer α)), 5.70 (dd, H-3), 2.64 (d, H−1β).

### Step 11.f: Preparation of methyl (3,6-di-O-acetyl-2-azido-2,4-dideoxy-4-(prop-1-en-1-yl)-α-D-gluco-pyranosyl)-(1-4)-(methyl 2-O-acetyl-3-O-benzyl-α-L-idopyranosyluronate)-(1-4)-(6-O-acetyl-3-O-benzyl-2-benzyloxycarbonylamino-2-deoxy-α-D-glucopyranoside) (No. 82)

Compound 80 (5.65 g, 12.3 mmol) obtained in step 11.e and compound 81 (prepared according to J. C. Jacquinet et al., Carbohydr. Res. 130 (1984), 221-241) (11.54 g, 1.2 molar equivalents) are reacted according to METHOD 2 to give, after purification, compound 82 (9.39 g, 71%).

Mass spectrum (ESI) m/z 1077.5 [(M+H)⁺].

### Step 11.g: Preparation of methyl (3,6-di-O-acetyl-2-azido-2,4-dideoxy-4-(1,2-dihydroxypropyl)-α-D-glucopyranosyl)-(1-4)-(methyl 2-O-acetyl-3-O-benzyl-α-L-idopyranosyluronate)-(1-4)-(6-O-acetyl-3-O-benzyl-2-benzyloxycarbonylamino-2-deoxy-α-D-glucopyranoside) (No. 83)

N-Methylmorpholine N-oxide monohydrate (NMO) (1.11 g, 20 molar equivalents) and 4% osmium tetraoxide (OsO₄) in water (8.35 ml, 1 molar equivalent) are added to a solution of compound 82 (513. mg, 0.476 mmol) obtained in step 11.f, in a 1:1 tetrahydrofuran-dichloromethane mixture (8 ml). After stirring for 3 days at room temperature, a 1:1 dichloromethane-water mixture is added as well as a 37.5% sodium hydrogen sulphite (NaHSO₃) solution, and the stirring is maintained for an additional 30 min. The reaction mixture is extracted with dichloromethane, purified on silica and the fraction containing the starting material is allowed to react under the above conditions until it is completely consumed. After purification, compound 83 (293 mg, 66%) is finally obtained.

Mass spectrum (ESI) m/z 1111.4 [(M+H)⁺].

### Step 11.h: Preparation of methyl (3,6-di-O-acetyl-2-azido-2,4-dideoxy-4-(5-methyl-2-phenyl-1,3-dioxolan-4-yl)-α-D-glucopyranosyl)-(1-4)-(methyl 2-O-acetyl-3-O-benzyl-α-L-idopyranosyluronate)-(1-4)-(6-O-acetyl-3-O-benzyl-2-benzyloxycarbonylamino-2-deoxy-α-D-glucopyranoside) (No. 84)

CSA (21.6 mg, 0.2 mol equiv.) and benzylidene dimethyl acetal (160 μl, 2.3 molar equivalents) are added, under argon, to a solution of compound 83 (518 mg, 0.466 mmol) obtained in step 11.g, in acetonitrile. After stirring for 1 h 30 min, the medium is neutralized with triethylamine, concentrated to dryness and then purified on silica to give compound 84 (454 mg, 74%).

Mass spectrum (ESI) m/z 1199.5 [(M+H)⁺].

### Step 11.1: Preparation of methyl (2-azido-2,4-dideoxy-4-(5-methyl-2-phenyl-1,3-dioxolan-4-yl)-α-D-gluco-pyranosyl)-(1-4)-(3-O-benzyl-α-L-idopyranosyluronic acid)-(1-4)-(3-O-benzyl-2-benzyloxycarbonylamino-2-deoxy-α-D-glucopyranoside)(No. 85)

Compound 84 (215 mg, 0.18 mmol) obtained in step 11.h is treated according to method 3. After 16 h of magnetic stirring at room temperature, the medium is diluted with methanol (12.5 ml) and then a 4N aqueous sodium hydroxide solution (11.5 ml) is added at 0° C. The mixture is stirred for 4 h at 0° C., acidified (pH 5) with 6N hydrochloric acid and is extracted with dichloromethane, washed with 5% Na₂SO₃ and finally with saturated sodium chloride. After drying and concentrating, the residue is purified on silica to give compound 85 (157 mg, 86%).

Mass spectrum (ESI) m/z 1017.3 [(M+H)⁺].

### Step 11.j: Preparation of methyl (2-azido-2,4-dideoxy-4-(5-methyl-2-phenyl-1,3-dioxolan-4-yl)-3,6-di-O-sodium sulphonato-α-D-glucopyranosyl)-(1-4)-(sodium 3-O-benzyl-2-O-sodium sulphonato-α-L-idopyranosyluronate)-(1-4)-(3-O-benzyl-2-benzyloxycarbonylamino-2-deoxy-6-β-sodium sulphonato-α-D-glucopyranoside)(No. 86)

Compound 85 (160 mg, 0.157 mmol) obtained in step 11.i is treated according to METHOD 4 to give compound 86 (215 mg, 95%).

Mass spectrum (ESI) m/z 689.1 [(M+H−3Na)²⁻].

### Step 11.k: Preparation of methyl (2-amino-2,4-dideoxy-4-(1,2-dihydroxypropyl)-3,6-di-O-sodium sulphonato-α-D-glucopyranosyl)-(1-4)-(sodium 2-O-sodium sulphonato-α-L-idopyranosyluronate)-(1-4)-(2-amino-2-deoxy-6-O-sodium sulphonato-α-D-glucopyranoside) (No. 87)

Compound 86 (210 mg, 0.145 mmol) obtained in step 11.j is treated according to method 5 without acetic acid to give compound 87 (108 mg, 73%). If necessary, the reaction is repeated several times until the benzyl protons disappear completely by NMR.

Mass spectrum (ESI) m/z 996.1 [(M+H−Na)⁻].

### Step 11.1: Preparation of methyl (2-N-sodium sulphonato-2,4-dideoxy-4-(1,2-dihydroxypropyl)-3,6-di-O-sodium sulphonato-α-D-glucopyranosyl)-(1-4)-(sodium 2-O-sodium sulphonato-α-L-idopyranosyluronate)-(1-4)-(2-N-sodium sulphonato-2-deoxy-6-O-sodium sulphonato-α-D-glucopyranoside) (No. 88)

Pyridine.SO₃ complex (662 mg, 4.16 mmol) is added, at 0° C., to a solution of compound 87 (106 mg, 0.104 mmol) obtained in step 11.k, in water (7 ml), while the pH is kept at 9.3 with 1N sodium hydroxide. The temperature is then increased to room temperature, the reaction medium is stirred for 16 h while the pH is kept at 9.3, and is then purified on a Sephadex® G-25 gel column equilibrated with a 0.2M sodium chloride solution. After combining the fractions containing the product and concentrating, the residue is purified by the same Sephadex® G-25 column eluted with water, to give compound 88 (116 mg, 91%).

Mass spectrum (ESI) m/z 1199.8 [(M+H−Na)⁻].

### Step 11.m: Preparation of methyl (2-N-sodium sulphonato-2,4-dideoxy-4-formyl-3,6-di-O-sodium sulphonato-α-D-glucopyranosyl)-(1-4)-(sodium 2-O-sodium sulphonato-α-L-idopyranosyluronate)-(1-4)-(2-N-sodium sulphonato-2-deoxy-6-O-sodium sulphonato-α-D-glucopyranoside) (No. 89)

Sodium periodate (22.1 mg, 1.1 molar equivalents) is added to a solution of compound 88 (115 mg, 94 μmol) obtained in step 11.1, in water (1.9 ml). After 1 h of magnetic stirring, the reaction medium is purified on a Sephadex® G-15 gel column equilibrated in water to give compound 89 (107 mg, 96%).

### Preparation 12

### Synthesis of (2-acetamido-3,4-di-O-benzyl-2-deoxy-6-β-sodium sulphonato-α-D-glucopyranosyl)-(1-4)-(5-(benzyloxy)-4-oxypiperidine-1-carboxylate of benzyl-3-carboxylate of sodium (3S,4R,5R)) (No. 97)

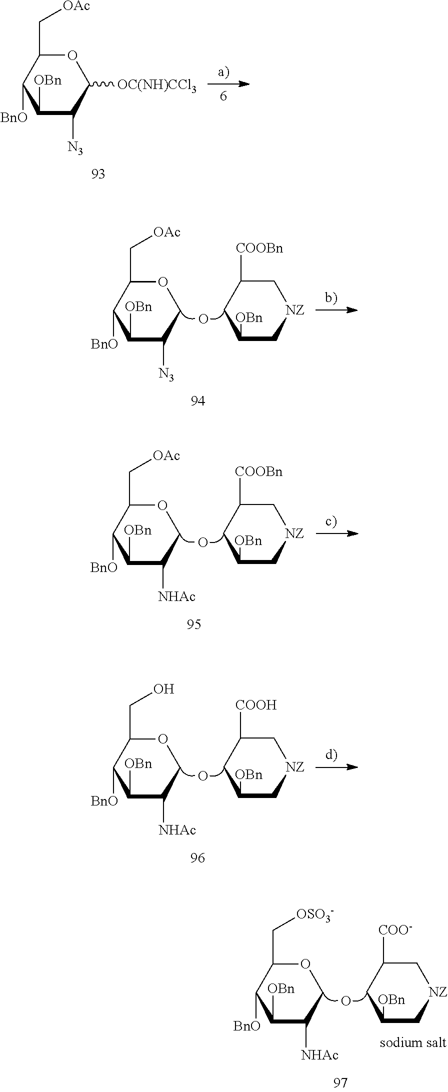

### Step 12.a: Preparation of (6-O-acetyl-2-azido-3,4-di-O-benzyl-2-deoxy-α-D-glucopyranosyl)-(1-4)-((3S,4R,5R)-dibenzyl 5-(benzyloxy)-4-oxypiperidine-1,3-dicarboxylate) (No. 94)

Compound 93 (prepared according to R. Verduyn et al., Recl. Tray. Chim. the Netherlands, 109 (1990), 12, 591) (361 mg, 0.631 mmol) and compound 6 (200 mg, 0.421 mmol) obtained in step 1.e are treated according to method 2 to give, after purification, compound 94 (224 mg, 60%).

Mass spectrum (ESI) m/z 885.4 [(M+H)⁺].

### Step 12.b: Preparation of (6-O-acetyl-2-acetamido-3,4-di-O-benzyl-2-deoxy-α-D-glucopyranosyl)-(1-4)-((3S,4R,5R)-dibenzyl 5-(benzyloxy)-4-oxypiperidine-1,3-dicarboxylate) (No. 95)

Compound 94 (56.3 mg, 63.6 μmol) obtained in step 12.a is treated as for the synthesis of 17 (step 3.f) to give compound 95 (54.5 mg, 95%). Mass spectrum (ESI) m/z 901.2 [(M+H)⁺].

### Step 12.c: Preparation of (2-acetamido-3,4-di-O-benzyl-2-deoxy-α-D-glucopyranosyl)-(1-4)-(3S,4R,5R)benzyl 5-(benzyloxy)-4-oxypiperidine-1-carboxylate-3-carboxylic acid (No. 96)

Compound 95 (101 mg, 50.9 μmol) obtained in step 12.b is treated according to METHOD 3. The residue is used in the raw state in the next step.

### Step 12.d: Preparation of (2-acetamido-3,4-di-O-benzyl-2-deoxy-6-O-sodium sulphonato-α-D-glucopyranosyl)-(1-4)-(5-(benzyloxy)-4-oxypiperidine-1-carboxylate of benzyl-3-carboxylate of sodium (3S,4R,5R))(No. 97)

The crude compound 96 obtained in step 12.c is treated according to METHOD 4, to give compound 97 (35 mg, 88% (2 steps)), which may be partially esterified on the carboxylic acid functional group.

Mass spectrum (ESI) m/z 847.2 [(M−H)⁻].

### Preparation 13

### Synthesis of (3R,4R,5R)-benzyl 3-(benzyloxy)-5-[(benzyloxy)methyl]-4-hydroxypiperidine-1-carboxylate (No. 100)

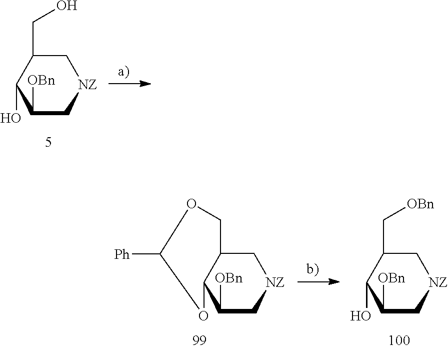

### Step 13.a: Preparation of (4aR,8R,8aR)benzyl 8-(benzyloxy)-2-phenyltetrahydro-4H-[1,3]dioxino[5,4-c]pyridine-6(5H)carboxylate (No. 99)

Camphorsulphonic acid (31 mg, 0.2 mol equiv.) and then benzaldehyde dimethyl acetal (0.23 ml, 2.3 mol equiv.) are added to a solution of compound 5 (250 mg, 0.67 mmol) in acetonitrile (13.4 ml). After 1 h of magnetic stirring at room temperature, the reaction medium is neutralized with triethylamine, concentrated and purified on silica gel (15:85 ethyl acetate-cyclohexane) to give compound 99 (281 mg, 91%). Mass spectrum (ESI) m/z 482.2 [(M+Na)⁺].

### Step 13.b: Preparation of (3R,4R,5R)-benzyl 3-(benzyloxy)-5-[(benzyloxy)methyl]-4-hydroxypiperidine-1-carboxylate (No. 100)

Triethylsilane (0.20 ml, 4 molar equivalents), trifluoroacetic acid (0.09 ml, 4 molar equivalents) and trifluoroacetic anhydride (3 μl, 0.07 molar equivalent) are successively added, at 0° C., under argon, to a solution of compound 99 (141 mg, 0.31 mmol) obtained in step 13.a, in dichloromethane (1.2 ml). The temperature is kept at 0° C. for 5 min and then the reaction medium is placed at room temperature for 3.5 h. The reaction mixture is then neutralized with an aqueous sodium hydrogen carbonate solution, with water, and the organic phase is dried (Na₂SO₄), filtered and concentrated under vacuum. The residue is purified on silica gel to give compound 100 (76 mg, 54%).

Mass spectrum (ESI) m/z 462.3 [(M+H)⁺].

### Preparation 14

### Synthesis of (3-O-benzyl-2,4-di-O-sodium sulphonato-α-L-idopyranosyluronate of sodium)-(1-4)-(2-acetamido-3-O-benzyl-2-deoxy-6-O-sodium sulphonato-α-D-gluco-pyranosyl)-(1-4)-(3-O-benzyl-2-O-sodium sulphonato-α-L-idopyranosyluronate of sodium)-(1-4)-(2-acetamido-3-β-benzyl-2-deoxy-6-O-sodium sulphonato-α-D-gluco-pyranosyl)-(1-4)-((3R,4R,5R)-benzyl 3-(benzyloxy)-5-[(benzyloxy)methyl]-4-oxypiperidine-1-carboxylate) (No. 104)

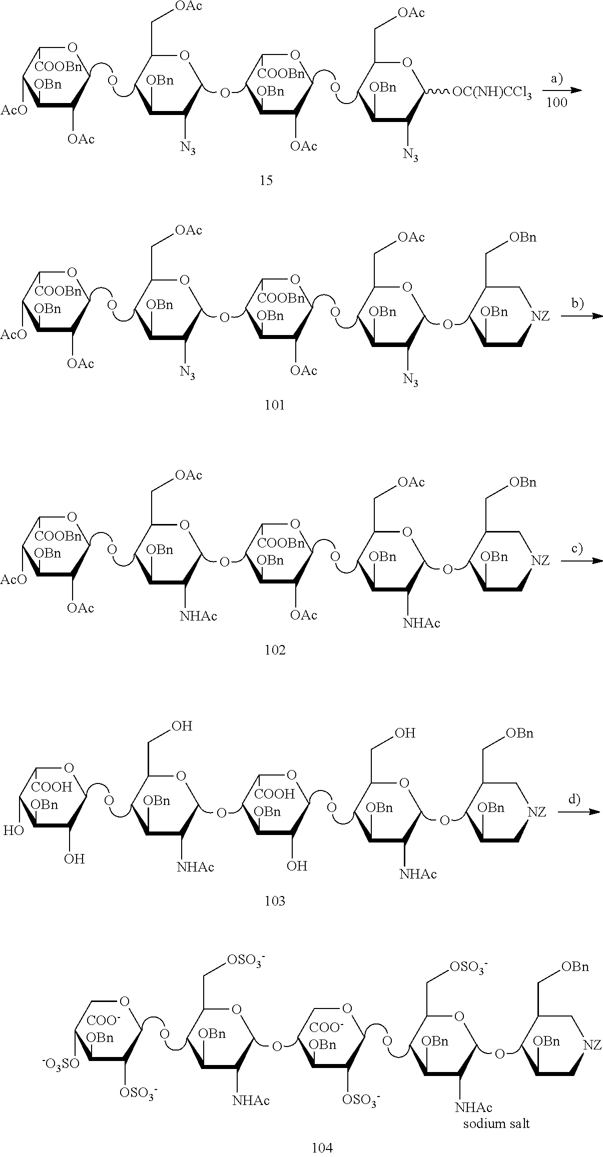

### Step 14.a: Preparation of (benzyl-2,4-di-O-acetyl-3-O-benzyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-O-benzyl-2-deoxy-α-D-gluco-pyranosyl)-((3R,4R,5R)-benzyl 3-(benzyloxy)-5-[(benzyl-oxy)methyl]-4-oxypiperidine-1-carboxylate) (No. 101)

Compounds 15 (123 mg, 0.075 mmol) and 100 (69 mg, 0.149 mmol) are treated according to METHOD 2 to give, after purification, compound 101 (117 mg, 80%). α/β ratio 55/45.

Mass spectrum (ESI) m/z 1962 [(M+Na)⁺].

### Step 14.b: Preparation of (benzyl 2,4-di-O-acetyl-3-O-benzyl-α-L-idopyranosyluronate)-(1-4)-(6-O-acetyl-2-acetamido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-acetamido-3-O-benzyl-2-deoxy-α-D-glucopyranosyl)-(1-4)-((3R,4R,5R)-benzyl 3-(benzyloxy)-5-[(benzyloxy)methyl]-4-oxypiperidine-1-carboxylate) (No. 102)

Compound 101 (117 mg, 60 μmol) is dissolved in pyridine (1 ml) and then thioacetic acid (1 ml, 225 molar equivalents) is added at 0° C. The reaction medium is stirred for 17 h at room temperature and is then concentrated and purified on silica gel (4:96 ethanol-toluene) to give compound 102 (50 mg, 42%). Mass spectrum (ESI) m/z 1971.9 [(M+H)⁺].

### Step 14.c: Preparation of 3-O-benzyl-α-L-idopyranosyluronic acid-(1-4)-(2-acetamido-3-O-benzyl-2-deoxy-α-D-glucopyranosyl)-(1-4)-(3-O-benzyl-α-L-idopyranosyluronic acid)-(1-4)-(2-acetamido-3-O-benzyl-2-deoxy-α-D-glucopyranosyl)-(1-4)-((3R,4R,5R)-benzyl 3-(benzyloxy)-5-[(benzyloxy)methyl]-4-oxypiperidine-1-carboxylate) (No. 103)

Compound 102 (50 mg, 25 μmol) is treated according to METHOD 3 to give the derivative 103.

Mass spectrum (ESI) m/z 1581.7 [(M+H)⁺].

### Step 14.d: Preparation of (3-O-benzyl-2,4-di-O-sodium sulphonato-α-L-idopyranosyluronate of sodium)-(1-4)-(2-acetamido-3-O-benzyl-2-deoxy-6-O-sodium sulphonato-α-D-glucopyranosyl)-(1-4)-(3-O-benzyl-2-O-sodium sulphonato-α-L-idopyranosyluronate of sodium)-(1-4)-(2-acetamido-3-O-benzyl-2-deoxy-6-O-sodium sulphonato-α-D-glucopyranosyl)-(1-4)-((3R,4R,5R)-benzyl 3-(benzyloxy)-5-[(benzyloxy)methyl]-4-oxypiperidine-1-carboxylate) (No. 104)

Compound 103 is treated according to METHOD 4, to give compound 104 (43 mg, 80%, (2 steps)). Mass spectrum (ESI) m/z 2134.3 [(M−Na)⁻].

### Preparation 15

### Synthesis of (benzyl 2-O-acetyl-3-O-benzyl-4-O-phenyl-propyl-α-L-idopyranosyluronate)-(1-4)-(6-O-acetyl-2-azido-3-O-benzyl-2-deoxy-α,β-D-glucopyranose trichloro-acetimidate) (No. 114)

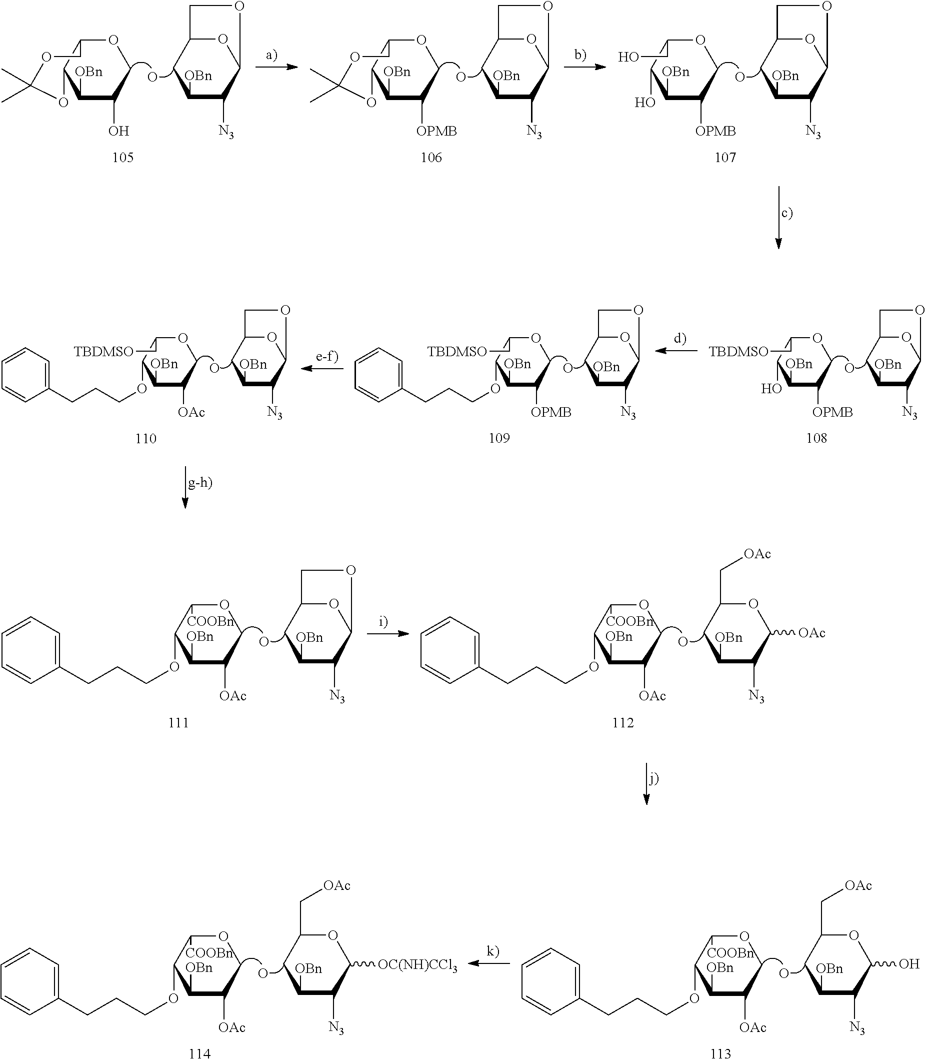

### Step 15.a: Preparation of (4,6-O-isopropylidene-3-β-benzyl-2-O-(4-methoxy)benzyl-α-L-idopyranosyl)-(1-4)-(1,6-anhydro-2-azido-3-O-benzyl-2-deoxy-β-D-glucopyranose) (No. 106)

NaH (6.93 g, 1.3 molar equivalents) and then para-methoxybenzyl chloride (24 ml, 1.6 molar equivalents) are added, at 0° C. and under argon, to a solution of compound 105 (63.2 g, 111 mmol) (prepared according to C. A. A. van Boeckel et al., J. Carbohydrate Chemistry (1985) 4 (3), 293-321) in DMF (445 ml). After 2 h of magnetic stirring, methanol is added (9 ml), the reaction medium is concentrated under vacuum, the crude reaction product is diluted with ethyl acetate, washed with water, dried (Na₂SO₄), filtered and concentrated. The residue obtained is purified on silica (ethyl acetate-cyclohexane 15:85) to give 106.

Mass spectrum (ESI) m/z 707.3 [(M+NH₄)⁺].

### Step 15.b: Preparation of (3-O-benzyl-2-O-(4-methoxy)benzyl-α-L-idopyranosyl)-(1-4)-(1,6-anhydro-2-azido-3-O-benzyl-2-deoxy-β-D-glucopyranose) (No. 107)

Compound 106 obtained in the preceding step is exposed to acetic acid at 80% in water. After 15 h of magnetic stirring, the reaction mixture is cooled with ice, diluted (dichloromethane) and neutralized with sodium hydrogen carbonate. The organic phase is dried (Na₂SO₄), filtered and concentrated. The residue obtained is purified on silica (ethyl acetate-cyclohexane 3:7) to give 107 (63.2 g, 88%, 2 steps). Mass spectrum (ESI) m/z 672.3 [(M+Na)⁺].

### Step 15.c: Preparation of (3-O-benzyl-6-O-tert-butyldimethylsilyl-2-O-(4-methoxy)benzyl-α-L-idopyranosyl)-(1-4)-(1,6-anhydro-2-azido-3-O-benzyl-2-deoxy-(3-D-glucopyranose) (No. 108)

Compound 107 (64.2 g) is dissolved in dichloromethane (200 ml). Triethylamine (30.3 ml, 2.2 molar equivalents), 4-dimethylaminopyridine (1.21 g, 0.1 molar equivalent) and tert-butyldimethylsilyl chloride (17.04 g, 1.1 molar equivalents) are successively added at 0° C. and under argon. After 4 h of magnetic stirring, 10% tert-butyldimethylsilyl chloride is added and after one hour, the reaction medium is diluted with dichloromethane, washed with water, dried (Na₂SO₄), filtered and concentrated. The residue obtained is purified on silica (ethyl acetate-cyclohexane 15:85) to give 108.

Mass spectrum (ESI) m/z 786.3 [(M+Na)⁺].

### Step 15.d: Preparation of (3-O-benzyl-6-O-tert-butyldimethylsilyl-2-O-(4-methoxy)benzyl-4-O-phenylpropyl-α-L-idopyranosyl)-(1-4)-(1,6-anhydro-2-azido-3-O-benzyl-2-deoxy-β-D-glucopyranose) (No. 109)

Phenylpropyl bromide (74 ml, 5 molar equivalents) and then NaH (7 g, 1.5 molar equivalents) are added, at 0° C. and under argon, to a solution of compound 108 in dimethylformamide (485 ml). After 5.5 h of magnetic stirring, methanol is added (50 ml), the reaction medium is concentrated under vacuum, the crude reaction product is diluted with ethyl acetate, washed with water, dried (Na₂SO₄), filtered and concentrated. The residue obtained is purified on silica (ethyl acetate-cyclohexane 15:85) to give 109 (49.3 g, 58%, 2 steps).

Mass spectrum (ESI) m/z 904.3 [(M+Na)⁺].

### Step 15.e-f: Preparation of (2-O-acetyl-3-O-benzyl-6-β-tert-butyldimethylsilyl-4-O-phenylpropyl-α-L-idopyranosyl)-(1-4)-(1,6-anhydro-2-azido-3-O-benzyl-2-deoxy-β-D-glucopyranose) (No. 110)

Water (112 ml) is added to a solution of 109 (49.3 g, 55.9 mmol) in dichloromethane (2.2 l) followed, at 0° C., by DDQ (19.03 g, 1.5 molar equivalents). After stirring for 3 h at 0° C., a sodium hydrogen carbonate solution is added. The organic phase is dried (Na₂SO₄), filtered and concentrated. The residue obtained is dissolved in pyridine (335 ml) and then acetic anhydride (28 ml) and 4-dimethylaminopyridine (682 mg) are added. After 16 h of magnetic stirring, the reaction mixture is concentrated under vacuum and the residue obtained is purified on silica (ethyl acetate-cyclohexane 15:85) to give 110 (34.4 g, 77%, 2 steps).

Mass spectrum (ESI) m/z 826.4 [(M+Na)⁺].

### Step 15.d-h: Preparation of (benzyl 2-O-acetyl-3-O-benzyl-4-O-phenylpropyl-α-L-idopyranosyluronate)-(1-4)-(1,6-anhydro-2-azido-3-O-benzyl-2-deoxy-β-D-glucopyranose) (No. 111)

A 3.5M aqueous sulphuric acid solution (45 ml) containing chromic anhydride (10 g) is added, at 0° C., to a solution of 110 (25.17 g, 31.3 mmol) in acetone (1.46 l). After 3 h of magnetic stirring at 0° C., the reaction medium is diluted with dichloromethane, washed with water, dried, filtered and concentrated to give a crude reaction product which is used directly in the next step. The residue obtained above is dissolved in dimethylformamide (230 ml) and potassium hydrogen carbonate (16.7 g, 5 molar equivalents) and benzyl bromide (39.8 ml, 10 molar equivalents) are added. The reaction mixture is stirred for 16 h at room temperature and is then diluted with ethyl acetate, washed with water, dried, filtered, concentrated and purified on silica gel (ethyl acetate-toluene 1:4) to give compound 111 (22.6 g, 91%, 2 steps).

Mass spectrum (ESI) m/z 811.3 [(M+NH₄)⁺].

### Step 15.i: Preparation of (benzyl 2-O-acetyl-3-O-benzyl-4-O-phenylpropyl-α-L-idopyranosyluronate)-(1-4)-(1,6-di-O-acetyl-2-azido-3-O-benzyl-2-deoxy-α,β-D-glucopyranose) (No. 112)

Trifluoroacetic acid (TFA) (1.14 ml, 11 molar equivalents) is added, at 0° C., to a solution of compound 111 (1.11 g, 1.39 mmol) in acetic anhydride (13.2 ml, 100 molar equivalents). After returning to room temperature, the reaction mixture is stirred for 3.5 h and is then concentrated, coevaporated with toluene and purified on silica gel (85:15 toluene-ethyl acetate) to give compound 112 (1.15 g, 93%).

Mass spectrum (ESI) m/z 918.3 [(M+Na)⁺].

### Step 15.j: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α,β-D-glucopyranose) (No. 113)

Benzylamine (BnNH₂) (5.25 ml, 38 molar equivalents) is added, at 0° C., to a solution of compound 112 (1.13 g, 1.26 mmol) in diethyl ether (51 ml). After stirring for 5 h 15 min at room temperature, the medium is acidified with 1N HCl and is then extracted with diethyl ether, dried (Na₂SO₄), concentrated and purified on silica gel (35:65 ethyl acetate-cyclohexane) to give 113 (0.97 g, 90%). Mass spectrum (ESI) m/z 854.3 [(M+H)⁺].

### Step 15.k: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α,β-D-glucopyranose trichloroacetimidate) (No. 114)

Caesium carbonate (Cs₂CO₃) (0.583 g, 1.6 molar equivalents) and then trichloroacetonitrile (CCl₃CN) (0.56 ml, 5.0 molar equivalents) are added, under argon, to a solution of compound 113 (0.95 g, 1.12 mmol) in dichloromethane (21.2 ml). After stirring for 35 min, the reaction mixture is filtered and then concentrated. The residue is purified on silica gel (25:75 ethyl acetate-cyclohexane) to give 114 (995 mg, 90%).

Mass spectrum (ESI) m/z 1021.5 [(M+Na)⁺].

### Preparation 16

### Synthesis of (benzyl 3-O-benzyl-4-O-phenylpropyl-2-β-sodium sulphonato-α-L-idopyranosyluronate)-(1-4)-(2-acetamido-3-O-benzyl-2-deoxy-6-O-sodium sulphonato-α-D-glucopyranosyl)-(1-4)-(benzyl 3-O-benzyl-2-O-sodium sulphonato-α-L-idopyranosyluronate)-(1-4)-(2-acetamido-3-O-benzyl-2-deoxy-6-O-sodium sulphonato-α-D-glucopyranosyl)-(1-4)-((3S,4R,5R)-dibenzyl 5-(benzyloxy)-4-oxypiperidine-1,3-dicarboxylate) (No. 122)

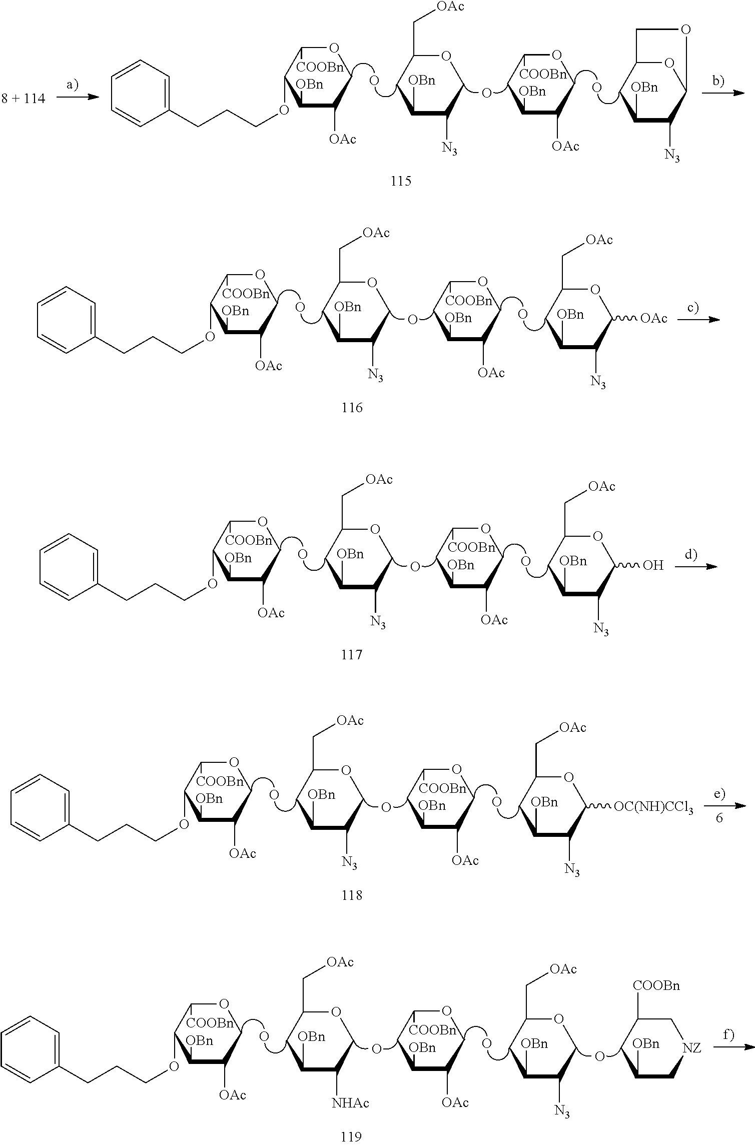

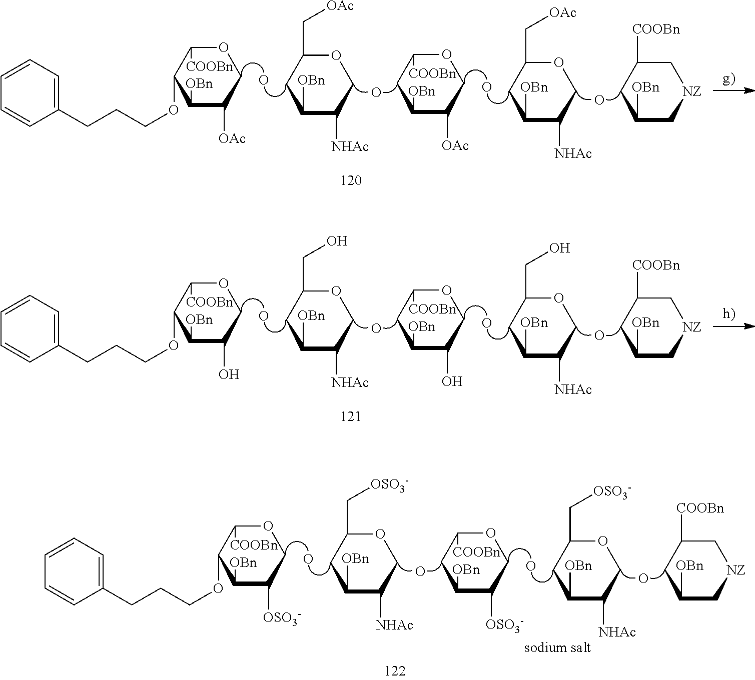

### Step 16.a: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(1,6-anhydro-2-azido-3-β-benzyl-2-deoxy-β-D-glucopyranose) (No. 115)

Compound 114 (990 mg, 0.99 mmol) and compound 8 (1.15 g, 1.7 mmol) are treated according to method 2 to give, after purification, compound 115 (623 mg, 42%).

Mass spectrum (ESI) m/z 1533.8 [(M+Na)⁺].

### Step 16.b: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(1,6-di-O-acetyl-2-azido-3-O-benzyl-2-deoxy-α,β-D-glucopyranose) (No. 116)

Compound 115 (590 mg, 0.39 mmol) is treated as for the synthesis of compound 112 to give, after purification on silica gel (7:3 cyclohexane-ethyl acetate), 116 (609 mg, 97%).

Mass spectrum (ESI) m/z 1636.2 [(M+Na)⁺].

### Step 16.c: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-β-benzyl-2-deoxy-α,β-D-glucopyranose) (No. 117)

Compound 116 (592 mg, 0.367 mmol) is treated as for the synthesis of compound 113 to give, after purification on silica gel (65:35 cyclohexane-ethyl acetate), compound 117 (530 mg, 92%).

Mass spectrum (ESI) m/z 1593.9 [(M+Na)⁺].

### Step 16.d: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-β-benzyl-2-deoxy-α,β-D-glucopyranose trichloroacetimidate) (No. 118)

Compound 117 (511 mg, 0.325 mmol) is treated as for the synthesis of compound 114 to give, after purification on silica gel (7:3 cyclohexane-ethyl acetate), 118 (495 mg, 89%).

Elemental analysis calculated for C₈₅H₉₀Cl₃N₇O₂₅: C, 59.49; H, 5.29; N, 5.71.

Found: C, 59.49; H, 5.50; N, 5.48.

### Step 16.e: Preparation of (benzyl 2-O-acetyl-3-O-benzyl-4-O-phenylpropyl-α-L-idopyranosyluronate)-(1-4)-(6-O-acetyl-2-azido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-azido-3-β-benzyl-2-deoxy-α-D-glucopyranosyl)-(1-4)-((3S,4R,5R)-dibenzyl 5-(benzyloxy)-4-oxypiperidine-1,3-dicarboxylate) (No. 119)

Compounds 118 (497 mg, 0.279 mmol) and 6 (255 mg, 0.536 mmol) are treated according to METHOD 2 to give, after purification, compound 119 (375 mg, 66%).

Elemental analysis calculated for C₁₁₁H₁₁₇N₇O₃₀: C, 59.49; H, 5.29; N, 5.71.

Found: C, 59.49; H, 5.50; N, 5.48.

### Step 16.f: Preparation of (benzyl 2-O-acetyl-3-O-benzyl-4-O-phenylpropyl-α-L-idopyranosyluronate)-(1-4)-(6-O-acetyl-2-acetamido-3-O-benzyl-2-deoxy-α-D-glucopyranosyl)-(1-4)-(benzyl 2-O-acetyl-3-O-benzyl-α-L-idopyranosyluronate)-(1-4)-(6-O-acetyl-2-acetamido-3-O-benzyl-2-deoxy-α-D-glucopyranosyl)-(1-4)-((3S,4R,5R)-dibenzyl 5-(benzyloxy)-4-oxypiperidine-1,3-dicarboxylate) (No. 120)

Compound 119 (180 mg, 88.7 μmol) is dissolved in pyridine (1.4 ml) and then thioacetic acid (1.4 ml, 225 molar equivalents) is added at 0° C. The reaction medium is stirred for 17 h at room temperature and is then concentrated and purified on silica gel (4:1 toluene-acetone) to give compound 120 (153 mg, 84%). Mass spectrum (ESI) m/z 2084.8 [(M+Na)⁺].

### Step 16.g: Preparation of (benzyl 3-O-benzyl-4-O-phenylpropyl-α-L-idopyranosyluronate)-(1-4)-(2-acetamido-3-O-benzyl-2-deoxy-α-D-glucopyranosyl)-(1-4)-(benzyl 3-O-benzyl-α-L-idopyranosyluronate)-(1-4)-(2-acetamido-3-O-benzyl-2-deoxy-α-D-glucopyranosyl)-(1-4)-((3S,4R,5R)-dibenzyl 5-(benzyloxy)-4-oxypiperidine-1,3-dicarboxylate) (No. 121)

Compound 120 (190 mg, 93.6 μmol) is treated according to METHOD 3. The polyol obtained is dissolved in dimethylformamide (4.4 ml), and potassium hydrogen carbonate (85 mg, 10 molar equivalents) and benzyl bromide (202 μl, 20 molar equivalents) are added at 0° C. The reaction mixture is stirred at room temperature for 16 h and is then purified on an LH-20 column.

Purification on silica gel (ethyl acetate-cyclohexane 2:3) makes it possible to obtain 121 (108 mg, 62% (2 steps)).

Mass spectrum (ESI) m/z 1884.2 [(M+Na)⁺].

### Step 16.h: Preparation of (benzyl 3-O-benzyl-4-O-phenylpropyl-2-O-sodium sulphonato-α-L-idopyranosyluronate)-(1-4)-(2-acetamido-3-O-benzyl-2-deoxy-6-O-sodium sulphonato-α-D-glucopyranosyl)-(1-4)-(benzyl 3-O-benzyl-2-O-sodium sulphonato-α-L-idopyranosyluronate)-(1-4)-(2-acetamido-3-O-benzyl-2-deoxy-6-O-sodium sulphonato-α-D-qlucopyranosyl)-(1-4)-((3S,4R,5R)-dibenzyl 5-(benzyloxy)-4-oxypiperidine-1,3-dicarboxylate) (No. 122)

Compound 121 (41 mg, 21.6 μmol) is treated according to METHOD 4 to give compound 122 (49 mg, 99%). Mass spectrum (ESI) m/z 2301.0 [(M−H)⁻].

The examples which follow illustrate the preparation of compounds of the invention without limiting it. The mass and NMR spectra confirm the structures of the compounds obtained.

### EXAMPLE 1

### Synthesis of (2,4-di-O-sodium sulphonato-α-L-idopyranosyluronate of sodium)-(1-4)-(2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-3-carboxylate of sodium (3S,4R,5R)) (compound No. 20)

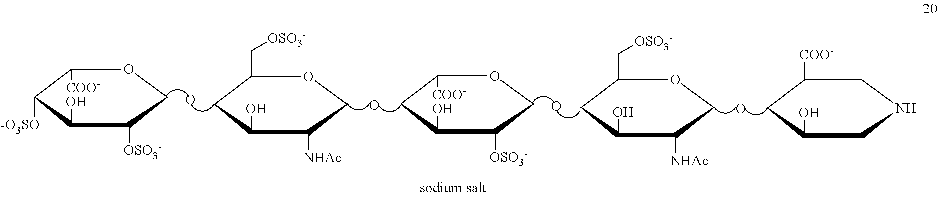

Compound 19 of PREPARATION 3 (50 mg, 24.02 μmol) is treated according to method 5 to give compound 20 (26 mg, 72%).

¹H NMR (D₂O) δ 5.23 (d, H−1 Glc^{II}), 5.13 (d, H−1 IdoUA^{III}), 5.10 (d, H−1 IdoUA^{V}), 5.09 (d, H−1 Glc^{IV}), 3.62, 3.04, 2.64, 2.47 (4m, 4H, H−2, H−2′, H−6, H−6′ pip^{I}).

### EXAMPLE 2

### Synthesis of (2-acetamido-2-deoxy-6-O-sodium sulphonato-α-D-glucopyranosyl)-(1-4)-(5-(hydroxy)-4-oxypiperidine-3-carboxylate of sodium (3S,4R,5R)) (compound No. 21)

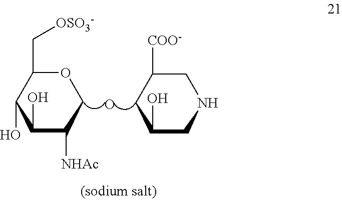

Compound 97 of PREPARATION 12 is treated according to method 5 to give compound 21.

¹H NMR (D₂O) δ 5.21 (d, H−1 Glc^{II}), 3.40, 3.40, 3.40, 3.10 (4m, 4H, H−2, H−2′, H−6, H−6′ pip^{I}).

### EXAMPLE 3

### Synthesis of (2,4-di-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-3-carboxylate of sodium (3S,4R,5R)) (compound No. 22)

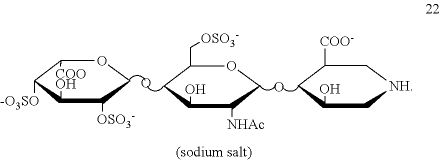

Compound 22 was prepared in the same manner. ¹H NMR (D₂O) δ 5.19 (d, H−1 Glc^{II}), 5.15 (d, H−1 IdoUA^{III}), 3.27, 3.23, 3.09, 2.89 (4m, 4H, H−2, H−2′, H-6, H−6′ pip^{I}).

### EXAMPLE 4

### Synthesis of (2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-3-carboxylate of sodium (3S,4R,5R)) (compound No. 23)

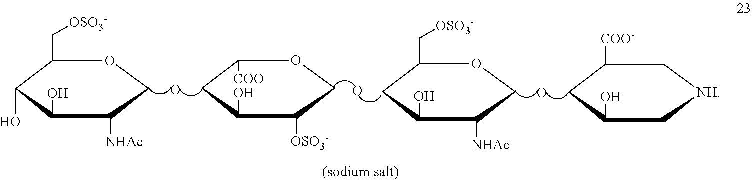

Compound 23 was prepared in the same manner. ¹H NMR (D₂O) δ 5.26 (d, H−1 Glc^{II}), 5.14 (d, H−1 IdoUA^{III}), 5.09 (d, H−1 Glc^{IV}).

### EXAMPLE 5

### Synthesis of (2,4-di-O-sodium sulphonato-α-L-idopyranosyluronate of sodium)-(1-4)-(2-N-sodium sulphonato-2-deoxy-6-O-sodium sulphonato-α-D-glucopyranosyl)-(1-4)-(2-O-sodium sulphonato-α-L-idopyranosyluronate of sodium)-(1-4)-(2-N-sodium sulphonato-2-deoxy-6-O-sodium sulphonato-α-D-glucopyranosyl)-(1-4)-(5-(hydroxy)-4-oxypiperidine-3-carboxylate of sodium (3S,4R,5R)) (compound No. 27)

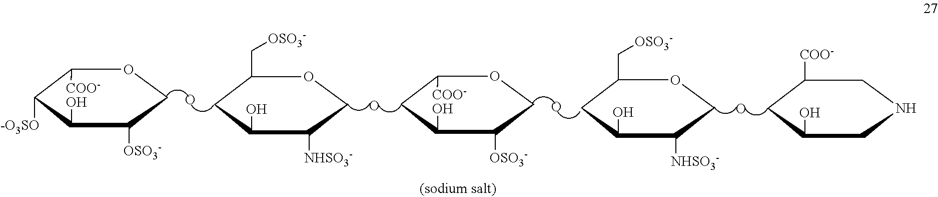

Compound 26 of PREPARATION 4 (7.0 mg, 3.28 μmol) is treated according to method 5 to give compound 27 (2.9 mg, 55%).

¹H NMR (D₂O) δ 5.53 (d, H−1 Glc^{II}), 5.44 (d, H−1 Glc^{IV}), 5.23 (d, H−1 IdoUA^{III}), 5.21 (d, H−1 IdoUA^{V}), 3.09, 3.07, 2.58, 2.44, (4m, 4H, H−2, H−2′, H−6, H−6′ pip^{I}).

### EXAMPLE 6

### Synthesis of (3-O-methyl-2,4-di-O-sodium sulphonato-α-L-idopyranosyluronate of sodium)-(1-4)-(3-O-methyl-2,6-di-O-sodium sulphonato-α-D-glucopyranosyl)-(1-4)-(3-O-methyl-2-O-sodium sulphonato-α-L-idopyranosyluronate of sodium)-(1-4)-(3-O-methyl-2,6-di-O-sodium sulphonato-α-D-glucopyranosyl)-(1-4)-(5-(hydroxy)-4-oxypiperidine-3-carboxylate of sodium (3S,4R,5R)) (compound No. 47)

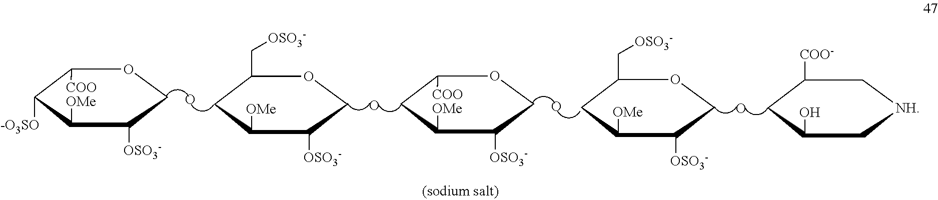

Compound 46 of PREPARATION 6 is treated according to method 5 to give, after purification, compound 47 (5 mg, 57%).

Mass spectrum (ESI) m/z 1650.9 [(M−Na+H)⁻].

### EXAMPLE 7

### Synthesis of (2,4-di-O-sodium sulphonato-α-L-idopyranosyluronate of sodium)-(1-4)-(2,6-di-O-sodium sulphonato-α-D-glucopyranosyl)-(1-4)-(2-O-sodium sulphonato-α-L-idopyranosyluronate of sodium)-(1-4)-(2,6-di-O-sodium sulphonato-α-D-glucopyranosyl)-(1-4)-(5-(hydroxy)-4-oxypiperidine-3-carboxylate of sodium (3S,4R,5R)) (compound No. 69)

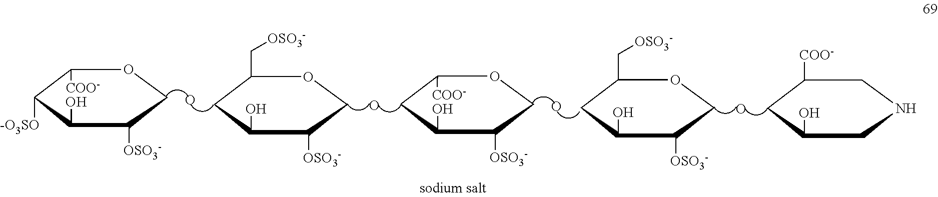

The crude compound 68 of PREPARATION 9 is treated according to METHOD 5 to give 69 (3.8 mg, 25%, two steps).

¹H NMR (D₂O) δ 5.62 (d, H−1 Glc^{IV}), 5.52 (d, H−1 Glc^{II}), 5.21 (d, H−1 IdoUA^{V}), 4.99 (d, H−1 IdoUA^{III}).

### EXAMPLE 8

### Synthesis of (4-O-propyl-2-O-sodium sulphonato-α-L-idopyranosyluronate of sodium)-(1-4)-(2,6-di-O-sodium sulphonato-α-D-glucopyranosyl)-(1-4)-(2-O-sodium sulphonato-α-L-idopyranosyluronate of sodium)-(1-4)-(2,6-di-O-sodium sulphonato-α-D-glucopyranosyl)-(1-4)-(5-(hydroxy)-4-oxypiperidine-3-carboxylate of sodium (3S,4R,5R)) (compound No. 74)

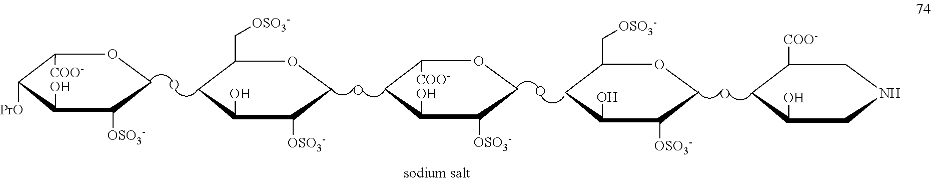

The crude compound 73 of PREPARATION 10 is treated according to METHOD 5 to give 74 (6.0 mg, 62%, two steps).

¹H NMR (D₂O) δ 5.68 (d, H−1 Glc^{IV}), 5.55 (d, H−1 Glc^{II}), 5.21 (d, H−1 IdoUA^{V}), 5.18 (d, H−1 IdoUA^{III}).

### EXAMPLE 9

### Synthesis of methyl (2,4-di-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-1-yl-3-carboxylate of sodium (3S,4R,5R))-(1-4)-(2-N-sodium sulphonato-2,4-dideoxy-4-methyl-3,6-di-O-sodium sulphonato-α-D-glucopyranosyl)-(1-4)-(sodium 2-O-sodium sulphonato-α-L-idopyranosyluronate)-(1-4)-(2-N-sodium sulphonato-2-deoxy-6-O-sodium sulphonato-α-D-glucopyranoside) (compound No. 90)

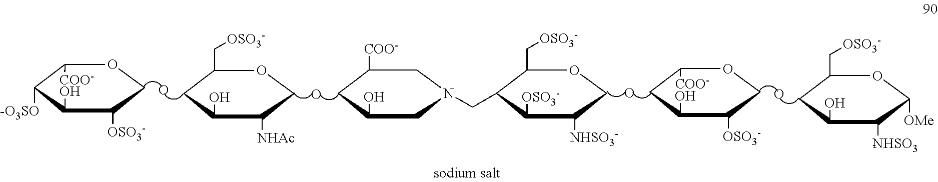

Sodium cyanoborohydride (4.4 mg, 2.4 mol equiv.) is added, at 0° C., to a solution of compound 89 (24 mg, 26.9 μmol) of PREPARATION 11, and of compound 22 (61 mg, 1.9 molar equivalents) of EXAMPLE 3 in phosphate buffer (pH 7). After stirring for 8 h at 0° C., the reaction medium is placed at room temperature for 16 h and then successively purified on a Sephadex® G-25 gel column eluted with 0.2M sodium chloride followed by the same Sephadex® G-25 column eluted with water to give compound 90 (17 mg, 31%).

Mass spectrum (ESI) m/z 2029.3 [(M+H−Na)⁻].

### EXAMPLE 10

### Synthesis of methyl (2,4-di-O-sodium sulphonato-α-L-idopyranosyluronate of sodium)-(1-4)-(2-acetamido-2-deoxy-6-O-sodium sulphonato-α-D-glucopyranosyl)-(1-4)-(5-(hydroxy)-4-oxypiperidine-1-yl-3-carboxylate of sodium (3S,4R,5R))-(1-4)-(2-N-sodium sulphonato-2,4-dideoxy-4-methyl-6-O-sodium sulphonato-α-D-glucopyranosyl)-(1-4)-(sodium 2-O-sodium sulphonato-α-L-idopyranosyluronate)-(1-4)-(2-N-sodium sulphonato-2-deoxy-6-O-sodium sulphonato-α-D-glucopyranoside) (compound No. 91)

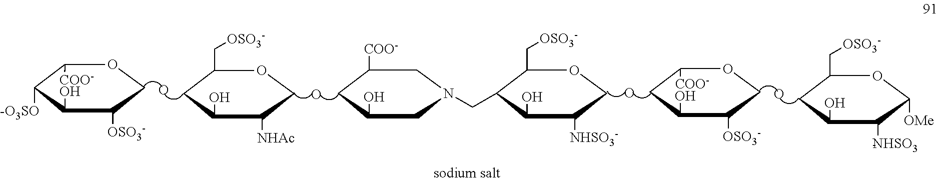

Compound 91 was prepared in the same manner. Mass spectrum (ESI) m/z 1927.1 [(M+H−Na)⁻].

### EXAMPLE 11

### Synthesis of methyl (5-(hydroxy-4-oxypiperidine-1-yl-3-carboxylate of sodium (3S,4R,5R))-(1-4)-(2-N-sodium sulphonato-2,4-dideoxy-4-methyl-3,6-di-O-sodium sulphonato-α-D-glucopyranosyl)-(1-4)-(sodium 2-O-sodium sulphonato-α-L-idopyranosyluronate)-(1-4)-(2-N-sodium sulphonato-2-deoxy-6-O-sodium sulphonato-α-D-glucopyranoside) (compound No. 92)

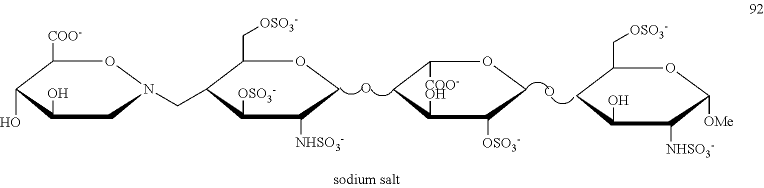

Compound 92 was prepared in the same manner. Mass spectrum (ESI) m/z 1321.1 [(M+H−Na)⁻].

### EXAMPLE 12

### Preparation of (2-acetamido-2-deoxy-6-O-sodium sulphonato-α-D-glucopyranosyl)-(1-4)-(5-(hydroxy-4-oxypiperidine-1-methyl-3-carboxylate of sodium (3S,4R,5R)) (compound No. 98)

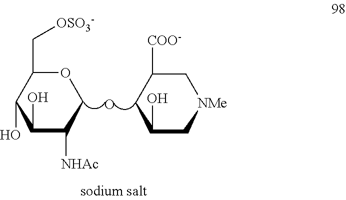

Compound 97 of PREPARATION 12 (20-mg, 21.3 μmol) is treated according to METHOD 5 in a 3:1:1 methanol-acetic acid-water mixture under a hydrogen stream to give compound 98 (6.0 mg, 57%)

Mass spectrum (ESI) m/z 456.8 [(M−H)⁻].

### EXAMPLE 13

### Synthesis of (2,4-di-O-sodium sulphonato-α-L-idopyranosyluronate of sodium)-(1-4)-(2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1,4)-(5-(hydroxy)-3-hydroxymethyl-4-oxypiperidine-(3R,4R,5R)) (compound No. 123)

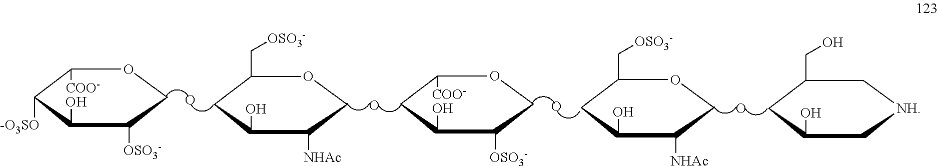

Compound 104 (41 mg, 19 μmol) is treated according to method 5 to give compound 123 (10.7 mg, 38%).

¹H NMR (D₂O) δ 5.28 (d, H−1 Glc^{II}), 5.20 (d, H−1 IdoUA^{III}), 5.17 (d, H−1 IdoUA^{V}), 5.15 (d, H−1 Glc^{IV}), 3.21, 3.19, 2.68, 2.59 (4m, 4H, H−2, H−2′, H−6, H−6′ pip^{I}).

### EXAMPLE 14

### Synthesis of (4-O-phenylpropyl-2-O-sodium sulphonato-α-L-idopyranosyluronate of sodium)-(1-4)-(2-acetamido-2-deoxy-6-O-sodium sulphonato-α-D-glucopyranosyl)-(1-4)-(2-O-sodium sulphonato-α-L-idopyranosyluronate of sodium)-(1-4)-(2-acetamido-2-deoxy-6-O-sodium sulphonato-α-D-glucopyranosyl)-(1,4)-(5-(hydroxy)-4-oxypiperidine-3-carboxylate of sodium (3R,4R,5R)) (compound No. 124)

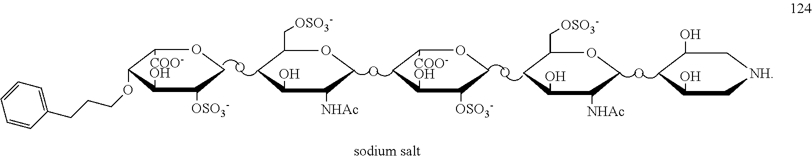

Compound 122 is treated according to method 5 to give compound 124.

¹H NMR (D₂O) δ 5.28 (d, H−1 Glc^{II}), 5.21 (d, H−1 IdoUA^{V}), 5.16 (d, H−1 IdoUA^{III}), 5.16 (d, H−1 Glc^{IV}), 3.23, 3.20, 2.93, 2.75 (4m, 4H, H−2, H−2′, H−6, H−6′ pip^{I}).

What is claimed is:

1. A compound of general formula (I): 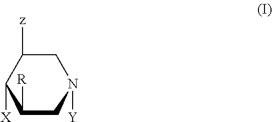
in which:
R represents a hydrogen atom, a hydroxyl radical, an —OSO₃⁻ radical, an —O—(C₁-C₅)alkyl radical or an —O-aralkyl radical;
Z represents a COO⁻ radical or a hydroxyl radical;
X represents a hydroxyl radical or a saccharide unit of formula A: 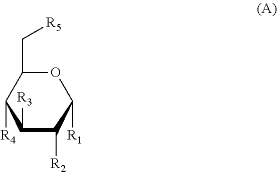
in which:
R₁ represents an oxygen atom, allowing A to bind to the azasugar unit or to another saccharide unit,
R₂ represents an —NH₂ radical, an —NHCO(C₁-C₅)-alkyl radical, an —NHCOaryl radical, an —NHSO₃⁻ radical, a hydroxyl radical, an —O—(C₁-C₅)alkyl radical, an —O-aralkyl radical or an —OSO₃⁻ radical,
R₃ represents a hydroxyl radical, an —OSO₃⁻ radical, an —O—(C₁-C₅)alkyl radical or an —O-aralkyl radical,
R₄ represents a hydroxyl radical, an —OSO₃⁻ radical, an —O—(C₁-C₅)alkyl radical, an —O-aralkyl radical or a saccharide unit of formula B: 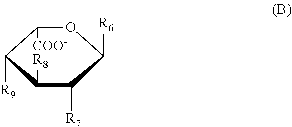
in which:
R₆ represents an oxygen atom, allowing B to bind to another saccharide unit of formula A,
R₇ and R₈ have the same definition as R₃ as defined above,
R₉ represents a hydroxyl group, an —OSO₃⁻ radical, an —O—(C₁-C₅)alkyl radical, an —O-aralkyl radical or a saccharide unit of formula A as defined above,
R₅ has the same definition as R₃ as defined above;
Y represents a hydrogen atom, a (C₁-C₅)alkyl radical or a saccharide unit of formula D 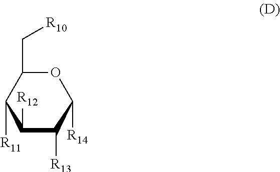
in which:
R₁₀, R₁₂ and R₁₃ have the same definitions as R₅, R₃ and R₂ respectively as defined above,
R¹¹ represents:
a (C₁-C₃)alkylene radical allowing D to attach to the azasugar unit, or
an oxygen atom allowing D to attach to another saccharide unit,
R₁₄ represents an —O— (C₁-C₅)alkyl radical or a radical of formula —O-E in which E represents a radical of formula: 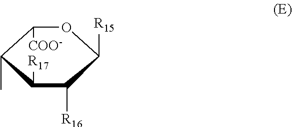
in which:
R₁₅ represents an —O— (C₁-C₅)alkyl radical, an —O-aralkyl radical or a saccharide unit of formula D in which R₁₁ represents an oxygen atom,
R₁₆ and R¹⁷ have the same definition as R₃ as defined above,
provided, however, that when X and R each represent a hydroxyl radical, Y does not represent a hydrogen atom, and it being understood that the number of saccharide units of which the compound of formula (I) is composed is between 1 and 10,
in free form or in the form of salts formed with a pharmaceutically acceptable base or acid, and in the form of solvates or hydrates.

2. The compound according to claim 1, of general formula (I): 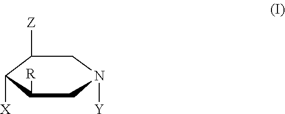
in which:
R represents a hydrogen atom, a hydroxyl radical, an —OSO₃⁻ radical, an —O—(C₁-C₅)alkyl radical or an —O-aralkyl radical;
Z represents a COO⁻ radical or a hydroxyl radical;
X represents a hydroxyl radical or a saccharide unit of formula A: 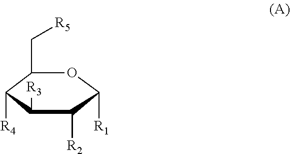
in which:
R₁ represents an oxygen atom,
R₂ represents an —NHCOCH₃ radical, an —NHSO₃⁻ radical, an —OSO₃⁻ radical,
R₃ represents a hydroxyl radical or an —O—(C₁-C₅)alkyl radical,
R₄ represents a hydroxyl radical, an —O-aralkyl radical or a saccharide unit of formula B: 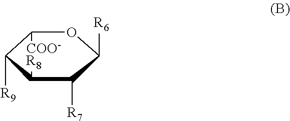
in which:
R₆ represents an oxygen atom,
R₇ represents an —OSO₃⁻ radical,
R₈ represents a hydroxyl radical, an —O—(C₁-C₅)alkyl radical or an —O-aralkyl radical,
R₉ represents an —OSO₃⁻ radical, an O-aralkyl radical, an —O—(C₁-C₅)alkyl radical or a saccharide unit of formula A as defined above,
R₅ represents an —OSO₃⁻ radical;
Y represents a hydrogen atom or a saccharide unit of formula D: 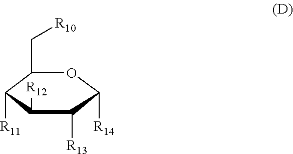
in which:
R₁₀ has the same definition as R₅ as defined above,
R₁₂ represents a hydroxyl radical or an —OSO₃⁻ radical,
R₁₃ represents an —NHSO₃⁻ radical,
R₁₁ represents a methylene radical linked to an azasugar unit or an oxygen atom linked to E,
R₁₄ an —OCH₃ radical or a radical of formula —O-E in which E represents a radical of formula: 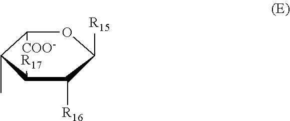
in which:
R₁₅ represents a D unit in which R₁₁ represents an oxygen atom allowing E to be linked to D,
R₁₆ represents an —OSO₃⁻ radical,
R₁₇ represents a hydroxyl radical,
it being understood that the number of saccharide units of which the compound of formula (I) is composed is between 2 and 10,
in free form or in the form of salts with a pharmaceutically acceptable base or acid, and in the form of solvates or hydrates.

3. The compound of general formula (I) according to claim 1, in which Y is a hydrogen atom and Z a COO⁻ radical.

4. A pharmaceutical composition comprising, as active ingredient, a compound of general formula (I) according to claim 1, in combination with at least one inert and appropriate excipient.
